SEQUENCE LISTING (239 sequences)

SEQ ID NO: 1 - RNA, 27 nt, synthetic construct
  source 1..27: mol_type = other RNA
taaaggaaccaattcagtcgactggat

SEQ ID NO: 2 - RNA, 39 nt, synthetic construct
  source 1..39: mol_type = other RNA
gccctgcaggtctagatatctcgagggtaccaaactgag

SEQ ID NO: 3 - RNA, 48 nt, synthetic construct
  source 1..48: mol_type = other RNA
cgggtctcacttggcgatgtcttggtctgcttgacaaagcaccagtgg

SEQ ID NO: 4 - RNA, 32 nt, synthetic construct
  source 1..32: mol_type = other RNA
taggtctcaaggtggtcattgcaccagccggg

SEQ ID NO: 5 - RNA, 38 nt, synthetic construct
  source 1..38: mol_type = other RNA
cgggtctccaccttcttttcagcgttttagagctagaa

SEQ ID NO: 6 - RNA, 54 nt, synthetic construct
  source 1..54: mol_type = other RNA
taggtctccaaacgcgatggagcgacagcaaacaaaaaaaaaagcaccgactcg

SEQ ID NO: 7 - RNA, 33 nt, synthetic construct
  source 1..33: mol_type = other RNA
cgggtctcacttggcgatgtcttggtctgcttg

SEQ ID NO: 8 - RNA, 33 nt, synthetic construct
  source 1..33: mol_type = other RNA
taggtctccaaacgcgatggagcgacagcaaac

SEQ ID NO: 9 - DNA, 24 bp, synthetic construct
  source 1..24: mol_type = other DNA
gaccaaagcctgtctatctgcatt

SEQ ID NO: 10 - DNA, 24 bp, synthetic construct
  source 1..24: mol_type = other DNA
agacgtcgacgatgaggatctcct

SEQ ID NO: 11 - DNA, 33 bp, synthetic construct
  source 1..33: mol_type = other DNA
gctcttccgatctccatgtcacccgaagccatg

SEQ ID NO: 12 - DNA, 33 bp, synthetic construct
  source 1..33: mol_type = other DNA
gctcttccgatctgcgactgcgagcacttacag

SEQ ID NO: 13 - DNA, 20 bp, synthetic construct
  source 1..20: mol_type = other DNA
aactatgattgctttgccgg

SEQ ID NO: 14 - DNA, 18 bp, synthetic construct
  source 1..18: mol_type = other DNA
ggcggccagcaattagca

SEQ ID NO: 15 - DNA, 22 bp, synthetic construct
  source 1..22: mol_type = other DNA
tgaaaataccattgcttggtca

SEQ ID NO: 16 - DNA, 21 bp, synthetic construct
  source 1..21: mol_type = other DNA
cacgtcaatgcagtccaaagc

SEQ ID NO: 17 - DNA, 23 bp, synthetic construct
  source 1..23: mol_type = other DNA
ctcggtgcgatatgaccgaataa

SEQ ID NO: 18 - DNA, 21 bp, synthetic construct
  source 1..21: mol_type = other DNA
ccacatcaatgtagtccatgc

SEQ ID NO: 19 - DNA, 21 bp, synthetic construct
  source 1..21: mol_type = other DNA
tcgaaaagttcgacagcgtct

SEQ ID NO: 20 - DNA, 21 bp, synthetic construct
  source 1..21: mol_type = other DNA
agctgcatcatcgaaattgcc

SEQ ID NO: 21 - DNA, 21 bp, synthetic construct
  source 1..21: mol_type = other DNA
aacggccacgagttcgagatc

SEQ ID NO: 22 - DNA, 20 bp, synthetic construct
  source 1..20: mol_type = other DNA
gctccacgatggtgtagtcc

SEQ ID NO: 23 - DNA, 23 bp, synthetic construct
  source 1..23: mol_type = other DNA
ctcgaaattttcccaaccgctcg

SEQ ID NO: 24 - DNA, 22 bp, synthetic construct
  source 1..22: mol_type = other DNA
acagagttggtgccgatgtcca

SEQ ID NO: 25 - DNA, 21 bp, synthetic construct
  source 1..21: mol_type = other DNA
atggttagttgatttaaaggg

SEQ ID NO: 26 - DNA, 21 bp, synthetic construct
  source 1..21: mol_type = other DNA
ctttgtcctaagctattacga

SEQ ID NO: 27 - DNA, 21 bp, synthetic construct
  source 1..21: mol_type = other DNA
aatgtttggttgtaagttaag

SEQ ID NO: 28 - DNA, 22 bp, synthetic construct
  source 1..22: mol_type = other DNA
tattacgggttattatcatcag

SEQ ID NO: 29 - DNA, 21 bp, synthetic construct
  source 1..21: mol_type = other DNA
aatgtttggttctaagttaac

SEQ ID NO: 30 - DNA, 21 bp, synthetic construct
  source 1..21: mol_type = other DNA
ctttgtcctaagctattacgg

SEQ ID NO: 31 - DNA, 29 bp, synthetic construct
  source 1..29: mol_type = other DNA
atgaccaccttcttttcagcaggtatgtc

SEQ ID NO: 32 - DNA, 27 bp, synthetic construct
  source 1..27: mol_type = other DNA
atgaccaccttctttagcaggtatgtc

SEQ ID NO: 33 - DNA, 10 bp, synthetic construct
  source 1..10: mol_type = other DNA
atggtatgtc

SEQ ID NO: 34 - DNA, 25 bp, synthetic construct
  source 1..25: mol_type = other DNA
atgaccaccttctagcaggtatgtc

SEQ ID NO: 35 - DNA, 25 bp, synthetic construct
  source 1..25: mol_type = other DNA
atgaccaccttcttttaggtatgtc

SEQ ID NO: 36 - DNA, 16 bp, synthetic construct
  source 1..16: mol_type = other DNA
atgaccacggtatgtc

SEQ ID NO: 37 - DNA, 30 bp, synthetic construct
  source 1..30: mol_type = other DNA
atgaccaccttcttttcaagcaggtatgtc

SEQ ID NO: 38 - DNA, 24 bp, synthetic construct
  source 1..24: mol_type = other DNA
atgaccacctttagcaggtatgtc

SEQ ID NO: 39 - DNA, 24 bp, synthetic construct
  source 1..24: mol_type = other DNA
atgaccaccttcagcaggtatgtc

SEQ ID NO: 40 - DNA, 30 bp, synthetic construct
  source 1..30: mol_type = other DNA
atgaccaccttcttttcgagcaggtatgtc

SEQ ID NO: 41 - DNA, 25 bp, synthetic construct
  source 1..25: mol_type = other DNA
atgaccaccttctagcaggtatgtc

SEQ ID NO: 42 - RNA, 23 nt, synthetic construct
  source 1..23: mol_type = other RNA
atgaccaccttcttttcagcagg

SEQ ID NO: 43 - DNA, 23 bp, synthetic construct
  source 1..23: mol_type = other DNA
acaacagggtgatcgtgcgcggg

SEQ ID NO: 44 - DNA, 24 bp, synthetic construct
  source 1..24: mol_type = other DNA
acaacagggtgatcgtgacgcggg

SEQ ID NO: 45 - DNA, 24 bp, synthetic construct
  source 1..24: mol_type = other DNA
acaacagggtgatcgtggcgcggg

SEQ ID NO: 46 - DNA, 46 bp, synthetic construct
  source 1..46: mol_type = other DNA
gttccacggcgagctctggcactgcatagggtacggtaccgggtac

SEQ ID NO: 47 - DNA, 46 bp, synthetic construct
  source 1..46: mol_type = other DNA
gttccacggcgagctctggcactgcatagggtacggcaccgggtac

SEQ ID NO: 48 - DNA, 46 bp, synthetic construct; identical to SEQ ID NO: 47
  source 1..46: mol_type = other DNA

SEQ ID NO: 49 - DNA, 46 bp, synthetic construct; identical to SEQ ID NO: 46
  source 1..46: mol_type = other DNA

SEQ ID NO: 50 - DNA, 19 bp, synthetic construct
  source 1..19: mol_type = other DNA
gttctacggcaccgggtac

SEQ ID NO: 51 - DNA, 23 bp, synthetic construct
  source 1..23: mol_type = other DNA
gttccacgtacggcaccgggtac

SEQ ID NO: 52 - DNA, 19 bp, synthetic construct
  source 1..19: mol_type = other DNA
gttctacggcaccgggtac

SEQ ID NO: 53 - DNA, 23 bp, synthetic construct
  source 1..23: mol_type = other DNA
gttccacgtacggcaccgggtac

SEQ ID NO: 54 - DNA, 46 bp, synthetic construct; identical to SEQ ID NO: 47
  source 1..46: mol_type = other DNA

SEQ ID NO: 55 - DNA, 46 bp, synthetic construct; identical to SEQ ID NO: 46
  source 1..46: mol_type = other DNA

SEQ ID NO: 56 - DNA, 46 bp, synthetic construct
  source 1..46: mol_type = other DNA
gttccacggcgagctctggcactgcatagggtacggcagcgggtac

SEQ ID NO: 57 - DNA, 46 bp, synthetic construct; identical to SEQ ID NO: 47
  source 1..46: mol_type = other DNA

SEQ ID NO: 58 - DNA, 46 bp, synthetic construct; identical to SEQ ID NO: 47
  source 1..46: mol_type = other DNA

SEQ ID NO: 59 - DNA, 46 bp, synthetic construct; identical to SEQ ID NO: 47
  source 1..46: mol_type = other DNA

SEQ ID NO: 60 - DNA, 45 bp, synthetic construct
  source 1..45: mol_type = other DNA
gttccacggcgagctctggcactgcataggtacggtaccgggtac

SEQ ID NO: 61 - DNA, 46 bp, synthetic construct; identical to SEQ ID NO: 56
  source 1..46: mol_type = other DNA

SEQ ID NO: 62 - DNA, 45 bp, synthetic construct; identical to SEQ ID NO: 60
  source 1..45: mol_type = other DNA

SEQ ID NO: 63 - DNA, 46 bp, synthetic construct; identical to SEQ ID NO: 46
  source 1..46: mol_type = other DNA

SEQ ID NO: 64 - DNA, 46 bp, synthetic construct; identical to SEQ ID NO: 56
  source 1..46: mol_type = other DNA

SEQ ID NO: 65 - DNA, 46 bp, synthetic construct; identical to SEQ ID NO: 47
  source 1..46: mol_type = other DNA

SEQ ID NO: 66 - DNA, 46 bp, synthetic construct; identical to SEQ ID NO: 56
  source 1..46: mol_type = other DNA

SEQ ID NO: 67 - DNA, 46 bp, synthetic construct; identical to SEQ ID NO: 56
  source 1..46: mol_type = other DNA

SEQ ID NO: 68 - DNA, 46 bp, synthetic construct; identical to SEQ ID NO: 47
  source 1..46: mol_type = other DNA

SEQ ID NO: 69 - DNA, 46 bp, synthetic construct; identical to SEQ ID NO: 46
  source 1..46: mol_type = other DNA

SEQ ID NO: 70 - DNA, 46 bp, synthetic construct; identical to SEQ ID NO: 56
  source 1..46: mol_type = other DNA

SEQ ID NO: 71 - DNA, 46 bp, synthetic construct; identical to SEQ ID NO: 47
  source 1..46: mol_type = other DNA

SEQ ID NO: 72 - DNA, 46 bp, synthetic construct; identical to SEQ ID NO: 56
  source 1..46: mol_type = other DNA

SEQ ID NO: 73 - DNA, 46 bp, synthetic construct; identical to SEQ ID NO: 46
  source 1..46: mol_type = other DNA

SEQ ID NO: 74 - DNA, 46 bp, synthetic construct; identical to SEQ ID NO: 56
  source 1..46: mol_type = other DNA

SEQ ID NO: 75 - DNA, 33 bp, synthetic construct
  source 1..33: mol_type = other DNA
gttccacggcgagctctgtacggcagcgggtac

SEQ ID NO: 76 - DNA, 46 bp, synthetic construct; identical to SEQ ID NO: 47
  source 1..46: mol_type = other DNA

SEQ ID NO: 77 - DNA, 46 bp, synthetic construct; identical to SEQ ID NO: 56
  source 1..46: mol_type = other DNA

SEQ ID NO: 78 - DNA, 46 bp, synthetic construct; identical to SEQ ID NO: 46
  source 1..46: mol_type = other DNA

SEQ ID NO: 79 - DNA, 46 bp, synthetic construct; identical to SEQ ID NO: 47
  source 1..46: mol_type = other DNA

SEQ ID NO: 80 - DNA, 33 bp, synthetic construct; identical to SEQ ID NO: 75
  source 1..33: mol_type = other DNA

SEQ ID NO: 81 - DNA, 46 bp, synthetic construct; identical to SEQ ID NO: 56
  source 1..46: mol_type = other DNA

SEQ ID NO: 82 - DNA, 42 bp, synthetic construct
  source 1..42: mol_type = other DNA
gttccacggcgagctctggcactgcagtacggcagcgggtac

SEQ ID NO: 83 - DNA, 46 bp, synthetic construct; identical to SEQ ID NO: 47
  source 1..46: mol_type = other DNA

SEQ ID NO: 84 - DNA, 46 bp, synthetic construct; identical to SEQ ID NO: 46
  source 1..46: mol_type = other DNA

SEQ ID NO: 85 - DNA, 46 bp, synthetic construct; identical to SEQ ID NO: 56
  source 1..46: mol_type = other DNA

SEQ ID NO: 86 - DNA, 46 bp, synthetic construct; identical to SEQ ID NO: 56
  source 1..46: mol_type = other DNA

SEQ ID NO: 87 - DNA, 42 bp, synthetic construct; identical to SEQ ID NO: 82
  source 1..42: mol_type = other DNA

SEQ ID NO: 88 - DNA, 46 bp, synthetic construct; identical to SEQ ID NO: 46
  source 1..46: mol_type = other DNA

SEQ ID NO: 89 - DNA, 45 bp, synthetic construct; identical to SEQ ID NO: 60
  source 1..45: mol_type = other DNA

SEQ ID NO: 90 - DNA, 46 bp, synthetic construct; identical to SEQ ID NO: 47
  source 1..46: mol_type = other DNA

SEQ ID NO: 91 - DNA, 46 bp, synthetic construct; identical to SEQ ID NO: 56
  source 1..46: mol_type = other DNA

SEQ ID NO: 92 - DNA, 46 bp, synthetic construct; identical to SEQ ID NO: 46
  source 1..46: mol_type = other DNA

SEQ ID NO: 93 - DNA, 46 bp, synthetic construct; identical to SEQ ID NO: 46
  source 1..46: mol_type = other DNA

SEQ ID NO: 94 - DNA, 23 bp, synthetic construct
  source 1..23: mol_type = other DNA
gttccacgtacggcaccgggtac

SEQ ID NO: 95 - DNA, 23 bp, synthetic construct
  source 1..23: mol_type = other DNA
gttccacgtacggcaccgggtac

SEQ ID NO: 96 - DNA, 46 bp, synthetic construct; identical to SEQ ID NO: 46
  source 1..46: mol_type = other DNA

SEQ ID NO: 97 - DNA, 46 bp, synthetic construct; identical to SEQ ID NO: 56
  source 1..46: mol_type = other DNA

SEQ ID NO: 98 - DNA, 46 bp, synthetic construct; identical to SEQ ID NO: 47
  source 1..46: mol_type = other DNA

SEQ ID NO: 99 - DNA, 45 bp, synthetic construct; identical to SEQ ID NO: 60
  source 1..45: mol_type = other DNA

SEQ ID NO: 100 - DNA, 46 bp, synthetic construct; identical to SEQ ID NO: 46
  source 1..46: mol_type = other DNA

SEQ ID NO: 101 - DNA, 46 bp, synthetic construct; identical to SEQ ID NO: 56
  source 1..46: mol_type = other DNA

SEQ ID NO: 102 - DNA, 46 bp, synthetic construct; identical to SEQ ID NO: 46
  source 1..46: mol_type = other DNA

SEQ ID NO: 103 - DNA, 46 bp, synthetic construct; identical to SEQ ID NO: 56
  source 1..46: mol_type = other DNA

SEQ ID NO: 104 - DNA, 46 bp, synthetic construct; identical to SEQ ID NO: 46
  source 1..46: mol_type = other DNA

SEQ ID NO: 105 - DNA, 45 bp, synthetic construct; identical to SEQ ID NO: 60
  source 1..45: mol_type = other DNA

SEQ ID NO: 106 - DNA, 19 bp, synthetic construct
  source 1..19: mol_type = other DNA
gttctacggcaccgggtac

SEQ ID NO: 107 - DNA, 33 bp, synthetic construct; identical to SEQ ID NO: 75
  source 1..33: mol_type = other DNA

SEQ ID NO: 108 - DNA, 42 bp, synthetic construct; identical to SEQ ID NO: 82
  source 1..42: mol_type = other DNA

SEQ ID NO: 109 - DNA, 19 bp, synthetic construct
  source 1..19: mol_type = other DNA
gttctacggcaccgggtac

SEQ ID NO: 110 - DNA, 46 bp, synthetic construct; identical to SEQ ID NO: 47
  source 1..46: mol_type = other DNA

SEQ ID NO: 111 - DNA, 23 bp, synthetic construct
  source 1..23: mol_type = other DNA
gttccacgtacggcaccgggtac

SEQ ID NO: 112 - DNA, 46 bp, synthetic construct; identical to SEQ ID NO: 47
  source 1..46: mol_type = other DNA

SEQ ID NO: 113 - DNA, 46 bp, synthetic construct; identical to SEQ ID NO: 46
  source 1..46: mol_type = other DNA

SEQ ID NO: 114 - DNA, 33 bp, synthetic construct; identical to SEQ ID NO: 75
  source 1..33: mol_type = other DNA

SEQ ID NO: 115 - DNA, 23 bp, synthetic construct
  source 1..23: mol_type = other DNA
gttccacgtacggcaccgggtac

SEQ ID NO: 116 - DNA, 49 bp, synthetic construct
  source 1..49: mol_type = other DNA
cacggcgagctctggcactgcatagggtacggtaccgggtacgggtagg

SEQ ID NO: 117 - DNA, 49 bp, synthetic construct
  source 1..49: mol_type = other DNA
cacggcgagctctggcactgcatagggtacggcaccgggtacgggtatg

SEQ ID NO: 118 - DNA, 49 bp, synthetic construct; identical to SEQ ID NO: 116
  source 1..49: mol_type = other DNA

SEQ ID NO: 119 - DNA, 49 bp, synthetic construct; identical to SEQ ID NO: 117
  source 1..49: mol_type = other DNA

SEQ ID NO: 120 - DNA, 45 bp, synthetic construct
  source 1..45: mol_type = other DNA
cacggcgagctctggcactgcggtacggcaccgggtacgggtatg

SEQ ID NO: 121 - DNA, 45 bp, synthetic construct; identical to SEQ ID NO: 120
  source 1..45: mol_type = other DNA

SEQ ID NO: 122 - DNA, 47 bp, synthetic construct
  source 1..47: mol_type = other DNA
cacggcgagctctggcactgcataggacggcaccgggtacgggtatg

SEQ ID NO: 123 - DNA, 47 bp, synthetic construct; identical to SEQ ID NO: 122
  source 1..47: mol_type = other DNA

SEQ ID NO: 124 - DNA, 48 bp, synthetic construct
  source 1..48: mol_type = other DNA
cacggcgagctctggcactgcataggtacggcaccgggtacgggtatg

SEQ ID NO: 125 - DNA, 48 bp, synthetic construct; identical to SEQ ID NO: 124
  source 1..48: mol_type = other DNA

SEQ ID NO: 126 - DNA, 49 bp, synthetic construct; identical to SEQ ID NO: 116
  source 1..49: mol_type = other DNA

SEQ ID NO: 127 - DNA, 48 bp, synthetic construct
  source 1..48: mol_type = other DNA
cacggcgagctctggcactgcataggtacggtaccgggtacgggtagg

SEQ ID NO: 128 - DNA, 47 bp, synthetic construct
  source 1..47: mol_type = other DNA
cacggcgagctctggcactgcatagtacggtaccgggtacgggtagg

SEQ ID NO: 129 - DNA, 48 bp, synthetic construct; identical to SEQ ID NO: 127
  source 1..48: mol_type = other DNA

SEQ ID NO: 130 - DNA, 47 bp, synthetic construct; identical to SEQ ID NO: 128
  source 1..47: mol_type = other DNA

SEQ ID NO: 131 - DNA, 45 bp, synthetic construct
  source 1..45: mol_type = other DNA
cacggcgagctctggcactgcggtacggcagcgggtacgggtatg

SEQ ID NO: 132 - DNA, 49 bp, synthetic construct; identical to SEQ ID NO: 117
  source 1..49: mol_type = other DNA

SEQ ID NO: 133 - DNA, 49 bp, synthetic construct; identical to SEQ ID NO: 117
  source 1..49: mol_type = other DNA

SEQ ID NO: 134 - DNA, 45 bp, synthetic construct; identical to SEQ ID NO: 131
  source 1..45: mol_type = other DNA

SEQ ID NO: 135 - DNA, 48 bp, synthetic construct; identical to SEQ ID NO: 124
  source 1..48: mol_type = other DNA

SEQ ID NO: 136 - DNA, 45 bp, synthetic construct; identical to SEQ ID NO: 120
  source 1..45: mol_type = other DNA

SEQ ID NO: 137 - DNA, 48 bp, synthetic construct; identical to SEQ ID NO: 124
  source 1..48: mol_type = other DNA

SEQ ID NO: 138 - DNA, 47 bp, synthetic construct; identical to SEQ ID NO: 122
  source 1..47: mol_type = other DNA

SEQ ID NO: 139 - DNA, 47 bp, synthetic construct; identical to SEQ ID NO: 122
  source 1..47: mol_type = other DNA

SEQ ID NO: 140 - DNA, 49 bp, synthetic construct; identical to SEQ ID NO: 117
  source 1..49: mol_type = other DNA

SEQ ID NO: 141 - DNA, 49 bp, synthetic construct; identical to SEQ ID NO: 117
  source 1..49: mol_type = other DNA

SEQ ID NO: 142 - DNA, 45 bp, synthetic construct; identical to SEQ ID NO: 120
  source 1..45: mol_type = other DNA

SEQ ID NO: 143 - DNA, 45 bp, synthetic construct
  source 1..45: mol_type = other DNA
cacggcgagctctggcactgcagtacggcagcgggtacgggtatg

SEQ ID NO: 144 - DNA, 45 bp, synthetic construct; identical to SEQ ID NO: 120
  source 1..45: mol_type = other DNA

SEQ ID NO: 145 - DNA, 45 bp, synthetic construct; identical to SEQ ID NO: 120
  source 1..45: mol_type = other DNA

SEQ ID NO: 146 - DNA, 48 bp, synthetic construct
  source 1..48: mol_type = other DNA
cacggcgagctctggcactgcataggtacggcagcgggtacgggtatg

SEQ ID NO: 147 - DNA, 49 bp, synthetic construct; identical to SEQ ID NO: 116
  source 1..49: mol_type = other DNA

SEQ ID NO: 148 - DNA, 49 bp, synthetic construct; identical to SEQ ID NO: 116
  source 1..49: mol_type = other DNA

SEQ ID NO: 149 - DNA, 47 bp, synthetic construct; identical to SEQ ID NO: 122
  source 1..47: mol_type = other DNA

SEQ ID NO: 150 - DNA, 48 bp, synthetic construct; identical to SEQ ID NO: 124
  source 1..48: mol_type = other DNA

SEQ ID NO: 151 - DNA, 49 bp, synthetic construct; identical to SEQ ID NO: 116
  source 1..49: mol_type = other DNA

SEQ ID NO: 152 - DNA, 49 bp, synthetic construct; identical to SEQ ID NO: 117
  source 1..49: mol_type = other DNA

SEQ ID NO: 153 - DNA, 48 bp, synthetic construct; identical to SEQ ID NO: 127
  source 1..48: mol_type = other DNA

SEQ ID NO: 154 - DNA, 45 bp, synthetic construct; identical to SEQ ID NO: 131
  source 1..45: mol_type = other DNA

SEQ ID NO: 155 - DNA, 49 bp, synthetic construct
  source 1..49: mol_type = other DNA
cacggcgagctctggcactgcatagggtacggcagcgggtacgggtatg

SEQ ID NO: 156 - DNA, 45 bp, synthetic construct; identical to SEQ ID NO: 131
  source 1..45: mol_type = other DNA

SEQ ID NO: 157 - DNA, 47 bp, synthetic construct; identical to SEQ ID NO: 122
  source 1..47: mol_type = other DNA

SEQ ID NO: 158 - DNA, 48 bp, synthetic construct; identical to SEQ ID NO: 146
  source 1..48: mol_type = other DNA

SEQ ID NO: 159 - DNA, 45 bp, synthetic construct; identical to SEQ ID NO: 143
  source 1..45: mol_type = other DNA

SEQ ID NO: 160 - DNA, 45 bp, synthetic construct; identical to SEQ ID NO: 131
  source 1..45: mol_type = other DNA

SEQ ID NO: 161 - DNA, 49 bp, synthetic construct
  source 1..49: mol_type = other DNA
cacggcgagctctggcactgcatagggtacggtaccgggtacgggtatg

SEQ ID NO: 162 - DNA, 48 bp, synthetic construct; identical to SEQ ID NO: 124
  source 1..48: mol_type = other DNA

SEQ ID NO: 163 - DNA, 48 bp, synthetic construct; identical to SEQ ID NO: 127
  source 1..48: mol_type = other DNA

SEQ ID NO: 164 - DNA, 45 bp, synthetic construct; identical to SEQ ID NO: 131
  source 1..45: mol_type = other DNA

SEQ ID NO: 165 - DNA, 49 bp, synthetic construct; identical to SEQ ID NO: 161
  source 1..49: mol_type = other DNA

SEQ ID NO: 166 - DNA, 47 bp, synthetic construct; identical to SEQ ID NO: 128
  source 1..47: mol_type = other DNA

SEQ ID NO: 167 - DNA, 47 bp, synthetic construct; identical to SEQ ID NO: 128
  source 1..47: mol_type = other DNA

SEQ ID NO: 168 - DNA, 45 bp, synthetic construct; identical to SEQ ID NO: 120
  source 1..45: mol_type = other DNA

SEQ ID NO: 169 - DNA, 45 bp, synthetic construct; identical to SEQ ID NO: 131
  source 1..45: mol_type = other DNA

SEQ ID NO: 170 - DNA, 49 bp, synthetic construct; identical to SEQ ID NO: 155
  source 1..49: mol_type = other DNA

SEQ ID NO: 171 - DNA, 49 bp, synthetic construct; identical to SEQ ID NO: 117
  source 1..49: mol_type = other DNA

SEQ ID NO: 172 - DNA, 47 bp, synthetic construct; identical to SEQ ID NO: 122
  source 1..47: mol_type = other DNA

SEQ ID NO: 173 - DNA, 45 bp, synthetic construct; identical to SEQ ID NO: 131
  source 1..45: mol_type = other DNA

SEQ ID NO: 174 - DNA, 49 bp, synthetic construct
  source 1..49: mol_type = other DNA
cacggcgagctctggcactgcatagggtacggcaccgggtacgggtagg

SEQ ID NO: 175 - DNA, 45 bp, synthetic construct; identical to SEQ ID NO: 131
  source 1..45: mol_type = other DNA

SEQ ID NO: 176 - DNA, 45 bp, synthetic construct; identical to SEQ ID NO: 143
  source 1..45: mol_type = other DNA

SEQ ID NO: 177 - DNA, 49 bp, synthetic construct; identical to SEQ ID NO: 117
  source 1..49: mol_type = other DNA

SEQ ID NO: 178 - DNA, 47 bp, synthetic construct; identical to SEQ ID NO: 122
  source 1..47: mol_type = other DNA

SEQ ID NO: 179 - DNA, 47 bp, synthetic construct; identical to SEQ ID NO: 128
  source 1..47: mol_type = other DNA

SEQ ID NO: 180 - DNA, 45 bp, synthetic construct; identical to SEQ ID NO: 120
  source 1..45: mol_type = other DNA

SEQ ID NO: 181 - DNA, 49 bp, synthetic construct; identical to SEQ ID NO: 117
  source 1..49: mol_type = other DNA

SEQ ID NO: 182 - DNA, 49 bp, synthetic construct; identical to SEQ ID NO: 161
  source 1..49: mol_type = other DNA

SEQ ID NO: 183 - DNA, 49 bp, synthetic construct; identical to SEQ ID NO: 116
  source 1..49: mol_type = other DNA

SEQ ID NO: 184 - DNA, 45 bp, synthetic construct; identical to SEQ ID NO: 143
  source 1..45: mol_type = other DNA

SEQ ID NO: 185 - DNA, 48 bp, synthetic construct; identical to SEQ ID NO: 146
  source 1..48: mol_type = other DNA

SEQ ID NO: 186 - DNA, 49 bp, synthetic construct; identical to SEQ ID NO: 116
  source 1..49: mol_type = other DNA

SEQ ID NO: 187 - DNA, 45 bp, synthetic construct; identical to SEQ ID NO: 143
  source 1..45: mol_type = other DNA

SEQ ID NO: 188 - DNA, 48 bp, synthetic construct; identical to SEQ ID NO: 127
  source 1..48: mol_type = other DNA

SEQ ID NO: 189 - DNA, 49 bp, synthetic construct; identical to SEQ ID NO: 116
  source 1..49: mol_type = other DNA

SEQ ID NO: 190 - DNA, 49 bp, synthetic construct; identical to SEQ ID NO: 155
  source 1..49: mol_type = other DNA

SEQ ID NO: 191 - DNA, 45 bp, synthetic construct
  source 1..45: mol_type = other DNA
cacggcgagctctggcactgcggtacggcaccgggtacgggtagg

SEQ ID NO: 192 - DNA, 48 bp, synthetic construct; identical to SEQ ID NO: 127
  source 1..48: mol_type = other DNA

SEQ ID NO: 193 - DNA, 47 bp, synthetic construct; identical to SEQ ID NO: 128
  source 1..47: mol_type = other DNA

SEQ ID NO: 194 - DNA, 45 bp, synthetic construct; identical to SEQ ID NO: 120
  source 1..45: mol_type = other DNA

SEQ ID NO: 195 - DNA, 45 bp, synthetic construct; identical to SEQ ID NO: 131
  source 1..45: mol_type = other DNA

SEQ ID NO: 196 - DNA, 49 bp, synthetic construct; identical to SEQ ID NO: 161
  source 1..49: mol_type = other DNA

SEQ ID NO: 197 - DNA, 49 bp, synthetic construct; identical to SEQ ID NO: 117
  source 1..49: mol_type = other DNA

SEQ ID NO: 198 - DNA, 45 bp, synthetic construct; identical to SEQ ID NO: 131
  source 1..45: mol_type = other DNA

SEQ ID NO: 199 - DNA, 45 bp, synthetic construct; identical to SEQ ID NO: 131
  source 1..45: mol_type = other DNA

SEQ ID NO: 200 - DNA, 48 bp, synthetic construct; identical to SEQ ID NO: 146
  source 1..48: mol_type = other DNA

SEQ ID NO: 201 - DNA, 47 bp, synthetic construct
  source 1..47: mol_type = other DNA
cacggcgagctctggcactgcataggacggcaccgggtacgggtagg

SEQ ID NO: 202 - RNA, 49 nt, synthetic construct
  source 1..49: mol_type = other RNA
tgatctgaagcctgccaatatattagtagacagttccatggtgcctaaa

SEQ ID NO: 203 - RNA, 47 nt, synthetic construct
  source 1..47: mol_type = other RNA
tgatctgaagcctgccaatattagtagacagttccatggtgcctaaa

SEQ ID NO: 204 - RNA, 48 nt, synthetic construct
  source 1..48: mol_type = other RNA
tgatctgaagcctgccaataattagtagacagttccatggtgcctaaa

SEQ ID NO: 205 - RNA, 49 nt, synthetic construct
  source 1..49: mol_type = other RNA
tgatctgaagcctgccaatattattagtagacagttccatggtgcctaa

SEQ ID NO: 206 - RNA, 44 nt, synthetic construct
  source 1..44: mol_type = other RNA
tgatctgaagcctgccattagtagacagttccatggtgcctaaa

SEQ ID NO: 207 - RNA, 49 nt, synthetic construct
  source 1..49: mol_type = other RNA
tgatctgaagcctgccaataaattagtagacagttccatggtgcctaaa

SEQ ID NO: 208 - skipped

SEQ ID NO: 209 - RNA, 49 nt, synthetic construct; identical to SEQ ID NO: 202
  source 1..49: mol_type = other RNA

SEQ ID NO: 210 - RNA, 47 nt, synthetic construct; identical to SEQ ID NO: 203
  source 1..47: mol_type = other RNA

SEQ ID NO: 211 - RNA, 48 nt, synthetic construct
  source 1..48: mol_type = other RNA
tgatctgaagcctgccaattattagtagacagttccatggtgcctaaa

SEQ ID NO: 212 - RNA, 48 nt, synthetic construct; identical to SEQ ID NO: 204
  source 1..48: mol_type = other RNA

SEQ ID NO: 213 - RNA, 46 nt, synthetic construct
  source 1..46: mol_type = other RNA
tgatctgaagcctgccaatttagtagacagttccatggtgcctaaa

SEQ ID NO: 214 - RNA, 44 nt, synthetic construct; identical to SEQ ID NO: 206
  source 1..44: mol_type = other RNA

SEQ ID NO: 215 - RNA, 48 nt, synthetic construct
  source 1..48: mol_type = other RNA
tgatctgaagcctgccaatatttagtagacagttccatggtgcctaaa

SEQ ID NO: 216 - RNA, 49 nt, synthetic construct
  source 1..49: mol_type = other RNA
tgatctgaagcctgccaatactattagtagacagttccatggtgcctaa

SEQ ID NO: 217 - RNA, 49 nt, synthetic construct; identical to SEQ ID NO: 205
  source 1..49: mol_type = other RNA

SEQ ID NO: 218 - RNA, 49 nt, synthetic construct
  source 1..49: mol_type = other RNA
gatctgaagcctgccaataatattagtagacagttccatggtgcctaaa

SEQ ID NO: 219 - RNA, 43 nt, synthetic construct
  source 1..43: mol_type = other RNA
tgatctgaagcctgccaatatagacagttccatggtgcctaaa

SEQ ID NO: 220 - RNA, 44 nt, synthetic construct
  source 1..44: mol_type = other RNA
tgatctgaagcctgccaatagtagacagttccatggtgcctaaa

SEQ ID NO: 221 - RNA, 49 nt, synthetic construct
  source 1..49: mol_type = other RNA
gaagcctgccaattgacagttattagtagacagttccatggtgcctaaa

SEQ ID NO: 222 - RNA, 45 nt, synthetic construct
  source 1..45: mol_type = other RNA
tgatctgaagcctgccaataagtagacagttccatggtgcctaaa

SEQ ID NO: 223 - RNA, 16 nt, synthetic construct
  source 1..16: mol_type = other RNA
tgatctgaagcctaaa

SEQ ID NO: 224 - RNA, 49 nt, synthetic construct; identical to SEQ ID NO: 202
  source 1..49: mol_type = other RNA

SEQ ID NO: 225 - RNA, 41 nt, synthetic construct
  source 1..41: mol_type = other RNA
tgatctgaagcctgctagtagacagttccatggtgcctaaa

SEQ ID NO: 226 - RNA, 42 nt, synthetic construct
  source 1..42: mol_type = other RNA
tgatctgaagcctgccaagtagacagttccatggtgcctaaa

SEQ ID NO: 227 - RNA, 20 nt, synthetic construct
  source 1..20: mol_type = other RNA
acaacagggtgatcgtccgt

SEQ ID NO: 228 - RNA, 20 nt, synthetic construct
  source 1..20: mol_type = other RNA
ggaactgtctactaatatat

SEQ ID NO: 229 - RNA, 20 nt, synthetic construct
  source 1..20: mol_type = other RNA
accataaaggggatttgtga

SEQ ID NO: 230 - RNA, 20 nt, synthetic construct
  source 1..20: mol_type = other RNA
atacaatggagaactagtta

SEQ ID NO: 231 - RNA, 20 nt, synthetic construct
  source 1..20: mol_type = other RNA
cttgcaaggacttgatgata

SEQ ID NO: 232 - RNA, 40 nt, synthetic construct
  source 1..40: mol_type = other RNA
gctcttccgatctagttcttcttacgtaagattgatcata

SEQ ID NO: 233 - RNA, 28 nt, synthetic construct
  source 1..28: mol_type = other RNA
gctcttccgatctggaaactgatttctc

SEQ ID NO: 234 - RNA, 33 nt, synthetic construct
  source 1..33: mol_type = other RNA
gctcttccgatcttgacagtgaattccgtaacc

SEQ ID NO: 235 - RNA, 35 nt, synthetic construct
  source 1..35: mol_type = other RNA
gctcttccgatctgctgactacaaacagattgtcc

SEQ ID NO: 236 - RNA, 33 nt, synthetic construct
  source 1..33: mol_type = other RNA
gctcttccgatctcaggccttgcaccagatctt

SEQ ID NO: 237 - RNA, 28 nt, synthetic construct
  source 1..28: mol_type = other RNA
gctcttccgatctcaaaatccgccagtt

SEQ ID NO: 238 - RNA, 33 nt, synthetic construct
  source 1..33: mol_type = other RNA
gctcttccgatcttagtaatgtggagcaccttc

SEQ ID NO: 239 - RNA, 35 nt, synthetic construct
  source 1..35: mol_type = other RNA
gctcttccgatcttaactatttggtagcagtagcc

(19) United States
(12) Patent Application Publication
(10) Pub. No.: US 2025/0066803 A1
(43) Pub. Date: Feb. 27, 2025

(54) METHODS FOR IMPROVING PROTEIN DELIVERY TO PLANT CELLS BY BIOLISTICS

(71) Applicant: THE REGENTS OF THE UNIVERSITY OF CALIFORNIA, Oakland, CA (US)

(72) Inventors: Myeong-Je Cho, Oakland, CA (US); Snigdha Poddar, Oakland, CA (US); Brian J. Staskawicz, Oakland, CA (US)

(73) Assignee: THE REGENTS OF THE UNIVERSITY OF CALIFORNIA, Oakland, CA (US)

(21) Appl. No.: 18/724,911

(22) PCT Filed: Dec. 29, 2022
(86) PCT No.: PCT/US2022/054260

§ 371 Date: Jun. 27, 2024

Related U.S. Application Data
(60) Provisional application No. 63/295,348, filed on Dec. 30, 2021.

(51) Int. Cl. C12N 15/82; C12N 5/04; C12N 9/22; C12N 15/11

(52) U.S. Cl. CPC C12N 15/8207; C12N 5/04; C12N 9/22; C12N 15/111; C12N 15/8213; C12N 2310/20

(57) ABSTRACT

Direct protein delivery to plant cells is a useful tool for both fundamental and applied research in plant science. In certain embodiments, the present invention provides a method of delivering a substance to plant tissue comprising biolistically delivering microparticles coated with the substance to the plant tissue to generate bombarded plant tissue.

### CROSS REFERENCE TO RELATED APPLICATION

This application claims priority to U.S. Provisional Application No. 63/295,348 filed on 30 Dec. 2021. The entire content of the application referenced above is hereby incorporated by reference herein.

### BACKGROUND

Direct protein delivery to plant cells is a useful tool for both fundamental and applied research in plant science. Even though protein can be expressed using a transgenic approach (e.g. through delivery of a nucleic acid encoding the protein), the protein of interest is sometimes modified for properties that cannot be achieved by its expression in the host plant organism, and some plant species or tissue types are recalcitrant to be transformed by standard methods known in the art. In addition, the protein expression requires a certain time period to be detected, and the expression level can be limited by several factors such as promoters, tissue types, incubation conditions, etc. Therefore, there remains a need for alternative methods for introducing materials into plant tissues.

### SUMMARY

In one aspect, provided herein is a method of biolistically delivering a substance to plant tissue comprising:
(a) applying a rupture or bombardment pressure of about 100 psi to about 3000 psi to a macrocarrier disk carrying at least one drop of coated microparticles in order to bombard the coated microparticles into the plant tissue to form bombarded plant tissue, wherein the microparticles are about 0.1 μm to about 2.0 μm in size and wherein the microparticles are in a volume of microparticle suspension of about 0.1 μl to about 50 μl; and
(b) culturing the bombarded plant tissue on growth media in optimal incubation conditions for each substance.

In one aspect, provided herein is a method of delivering a substance to plant tissue comprising:
(a) biolistically delivering microparticles coated with the substance to the plant tissue to generate bombarded plant tissue,
wherein the microparticles are biolistically delivered with a gene gun,
wherein 2 or more drops of microparticle suspension are spread over the inner circle of the macrocarrier disk before drying or lyophilization,
wherein the drops of microparticle suspensions are in a volume of about 0.1 μl to about 50 μl,
wherein the microparticles are about 0.1 μm to about 2.0 μm in size, and
wherein the biolistical delivering is performed at a rupture or bombardment pressure of about 100 psi to about 3000 psi, and
(b) culturing the bombarded plant tissue on growth media.

In a second aspect, provided herein a method of delivering RNPs to plant tissue comprising
(a) biolistically delivering microparticles coated with the substance to the plant tissue to generate bombarded plant tissue,
wherein the microparticles are biolistically delivered with a gene gun,
(b) the incubating of the bombarded plant tissue is performed at about 26° C. to about 40° C., and
(c) culturing the bombarded plant tissue on growth media.

### BRIEF DESCRIPTION OF DRAWINGS

FIGS. 1A-1C. Construct Schematics. Schematic diagrams of three transformation vectors used for Triticum aestivum L. cv. Fielder transformation. (FIG. 1A) pAct1IIPT-4 is a 5,870-bp plasmid containing hygromycin phosphotransferase (HPT) driven by the OsAct1 promoter and its intron. (FIG. 1B) pAct1IDsRED is a 5,139-bp plasmid containing DsRED driven by the OsAct1 promoter and its intron. (FIG. 1C) pRGE-PDS-PS2 is a 10,122-bp plasmid containing 2 gene cassettes where gRNA is driven by the Tau6 promoter and Cas9 driven by OsUbi10 promoter.

FIGS. 2A-2F. Stable wheat transformation via biolistics. Stable wheat transformation via gene gun schematic. FIG. 2A) Harvest immature wheat spikes 10-14 days post-anthesis.

FIG. 2B) Isolate immature embryos sized 1.7-2.2 mm and place on osmoticum medium for 4 hours. FIG. 2C) Shoot gold particles with gene gun and desired gold particle size and rupture disk pressure. FIG. 2D) Plant tissue is subjected to three rounds of callus induction media containing selection, subculturing every 3 weeks. FIG. 2E) Larger callus pieces derived from a single immature embryo are broken up and placed on regeneration medium for shoot formation. FIG. 2F) Plantlets that are at least 1 cm in height are transferred to rooting medium in Phytatrays™ and grown to size until they can be transferred to soil.

FIG. 3. Primer sets used for selection of transgenic plants and for mutation in genome-edited plants. Primer sequences (from top to bottom in FIG. 3) are SEQ ID NOs: 19-30.

FIGS. 4A-4D. DsRED Expression in Different Tissue Types in Transgenic Wheat. FIG. 4A) Transient expression and brightfield of an immature wheat embryo 3 days post bombardment. FIG. 4B) The formation of a dsRED sector growing 6-8 weeks post bombardment compared with brightfield image of the same sector. FIG. 4C) A stable transformation showing DsRED expression in leaf tissue in comparison to a wild-type control. FIG. 4D) A stably transformed dsRED plantlet 10-12 weeks post bombardment under fluorescence and brightfield.

FIG. 5. Stable transformation frequency at T₀ plant level using different bombardment parameters. Stable transformation frequencies for the three combinations of gold particle size and bombardment rupture pressure. Set 1 (0.4 μm) shows the transformation frequencies for 0.4 μm gold particles at rupture pressures of 650 psi, 900 psi and 1100 psi as well as the total transformation frequency of 0.4 μm as a whole. Set 2 (0.6 μm) shows the transformation frequencies for 0.6 μm gold particles at rupture pressures of 650 psi, 900 psi and 1100 psi as well as the total transformation frequency of 0.6 μm as a whole. Set 3 (1.0 μm) shows the transformation frequencies for 1.0 μm gold particles at rupture pressures of 650 psi, 900 psi and 1100 psi as well as the total transformation frequency of 1.0 μm as a whole.

FIGS. 6A-6B. Volume (weight) and surface area of different gold particle sizes. Different gold particles are capable of holding different quantities of DNA. FIG. 6A) The diameter of a sphere directly affects the number of gold particles by weight. As diameter increases, the number of gold particles in a fixed weight decreases. The smaller the gold particle the more particles will be available to be coated in DNA for each bombardment prep. FIG. 6B) Larger diameter directly affects surface area of each particle. The larger the diameter, the greater the surface area. The difference in diameter between 0.4 μm to 1.0 μm results in a 6.25-fold increase in surface area. This means that larger particles are capable of holding a greater amount of DNA.

FIGS. 7A-7B. Microcarrier size effect on transient DsRED expression and genome editing efficiency. (A) Photos were taken for transient DsRED expression 3 days post bombardment. (B) Sanger sequencing was performed to detect mutations in all 3 genomes of each transgenic event.

FIG. 8. Effect of temperature treatment on callus tissue quality and growth in wheat. Tissue culture heat treatment data. Table expressing the results of a 1-day osmoticum treatment followed by a 1-day temperature treatment of 10 embryos each and the final weight of the tissue after 35 days of callus induction expressed in grams.

FIG. 9. Mutation efficiency with four different temperature and three bombardment treatment in wheat.

FIG. 10. Molecular analysis of M₀ PDS knockout wheat plantlets showing albino phenotype. Molecular analysis of 2 albino phenotype PDS mutation events indicating biallelic mutations across all genomes and sequence. Nucleotide deletions are indicated by lined through letters in the ‘sequence’ column, while inserted or replacement nucleotides are underlined. The adjacent PAM site (AGG) is bolded and indicated by grey font. Mutations of deletion, insertion, or replacement are relative to the sequence of ATGACCACCTTCTTTTCAGCAGGTATGTC (SEQ ID NO:31). Sequences from top to bottom in FIG. 10 are SEQ ID NOs: 32-41. The gRNA sequence ATGACCACCTTCTTTTCAGCAGG is SEQ ID NO:42.

FIG. 11. Homozygous triple knockout mutant showing albino phenotype. Photos depicting albino PDS triple homozygous mutants in plates as well as in Phytatrays™. Albino event #1 produced as a result of the 34° C. 1-day heat treatment, shown in plate and Phytatray™ Albino event #2 produced as a result of the 34° C. 1 day heat treatment, shown in plate and Phytatray™.

FIG. 12. Phenotype of M₁ progeny plants derived from PC14A with monoallelic and biallelic mutations in the three different genomes. M₁ progeny segregation from heterozygous event PC14A which contained two monoallelic mutations and 1 biallelic mutation. Photo of 2 albino M₁ progeny of the 28 total plantlets.

FIG. 13. Phenotype and molecular analysis of M₁ progeny plants derived from PC14A. PC14A is an M₀ event with monoallelic and biallelic mutations in the 3 different genomes.

FIG. 14. Mutations in OsPi21 genes in the edited M₀ rice plants via RNP bombardment. The inserted nucleotides are underlined. The bolded nucleotides are a 3 nt protospacer adjacent motif (PAM) sequence (GGG) immediately 3′ of the target site. Sequences from top to bottom in FIG. 14 are SEQ ID NO: 43, SEQ ID NO: 44, SEQ ID NO: 45 and SEQ ID NO: 44.

FIG. 15. Amplicon sequencing results from 2 different M₀ wheat plant pools. Indels (deletions, substitutions and inserts) are indicated. Sequences from top to bottom in the left column of FIG. 15 (pool #2) are SEQ ID NOs: 46-115. Sequences from top to bottom in the right column of FIG. 15 (pool #16) are SEQ ID NOs: 116-201.

FIG. 16. Genotyping of eighteen M₀ wheat plants by Sanger sequencing. “+” indicates the presence of a mutation.

FIG. 17. Wheat protoplast viability curve, demonstrating decreased viability at higher temperatures. N=3. Error bars indicate SEM.

FIG. 18. Targeted editing efficiency of gRNA-Cas9 ribonucleoproteins (RNPs) with different temperature treatments in wheat protoplasts, demonstrating increased editing at 30° C. Five gRNAs, Pi21gD, Tsn1 g2, Tsn1 g3, Snn5 g1, and Snn5 g2 were tested and transfected independently into protoplasts. N=3. Error bars indicate SEM.

FIGS. 19A-19B. Cas9-RNP particle bombardment and temperature treatment of wheat immature embryos (IEs). (FIG. 19A) A schematic of the particle bombardment and editing efficiency assay pipeline. (FIG. 19B) Targeted editing efficiency of gRNA-Cas9 ribonucleoproteins (RNPs) with different temperature treatments in IEs across time points. Five gRNAs, Pi21gD, Tsn1 g2, Tsn1 g3, Snn5 g1, and Snn5 g2 were bombarded independently into IEs. Tissue pools at 14 dpb and 48 dpb consisted of 10 randomly chosen initially bombarded IEs. Editing efficiency for M₀ plants is based on aggregate data from all independently genotyped M₀ plants that emerged from 10 randomly chosen initially bombarded IEs. Percent tissue edited is defined as the percentage of tissue with insertions or deletions within 2 bp of the target cleavage site out of the total tissue pool.

FIG. 20. Summary of editing outcomes in Pi21, Tsn1, and Snn5 targeted M₀ wheat plants. Data for Pi21 is broken down by subgenome. A Subgenome is a subset of a genome, especially one that has a specific function. Tsn1 and Snn5 are only present on subgenome B. “% Tissue Edited” indicates the percentage of edited alleles among the total alleles analyzed from the M₀ pools. “% Plants Edited” indicates the percentage of plants with any level of editing among the total plants analyzed from the M₀ pools.

FIGS. 21A-21B. Cas9-RNP mediated editing in gold particle bombarded immature embryos IEs is sustained over time. (FIG. 21A) Quantification of the number of unique mutant alleles detected via deep sequencing. (FIG. 21B) Western blot detection of Cas9 in 10-IE bombarded samples taken 0, 2, 7, and 14 dpb with anti-Cas9 antibody. The top and bottom blot represent 2 independent sets of 10 IEs. +=9 ng (top) and 3 ng (bottom) Cas9; −=IEs that were not bombarded with Cas9-RNP; loading volume of 25 μl (top) and 40 μl (bottom) total soluble protein extract per IE sample.

FIG. 22. Example of the difference in the number of unique mutant alleles between 14 dpb and 48 dpb. Provided are the detected alleles in immature embryos of wheat bombarded with Tsn1 g2-Cas9 RNPs and treated at 37° C. The vertical bold dashed line represents the Cas9 cleavage site. Mutant alleles are marked with *. Wild type alleles are marked as WT. Dashes indicate base pair deletions, boxes indicate base pair insertions, and bold letters indicate base pair substitutions. Sequences from top to bottom in FIG. 22 are SEQ ID NOs:202-226.

FIGS. 23A-23B. Correlation plot between targeted editing efficiency of gRNA-Cas9 RNPs in protoplasts and immature embryos (IEs) of wheat at (FIG. 23A) 48 dpb and in (FIG. 23B) M₀ plants treated at 30° C.

FIGS. 24A-24M. SnToxA assay in Tsn1targeted M₀ wheat regenerants. (FIG. 24A) Fielder control grown from seed. (FIGS. 24B-M) independent M₀ regenerants with (FIGS. 24B, C) homozygous wildtype; (FIGS. 23D-H) heterozygous (FIG. 24D) −2; (FIG. 24E) −5; (FIG. 24F) −31; (FIG. 24G) −1; (FIG. 24H) +1; and (FIGS. 24I-M) biallelic or homozygous mutant (FIG. 24I) −2, −5; (FIG. 24J) −2, −2, (FIG. 24K) −1, −1; (FIG. 24L) −2, −2; (FIG. 24M) −1, −1 genotypes. Mutation notation is as follows: a positive number, +, indicates the number of bases inserted, a negative number, −, indicates the number of bases deleted.

FIGS. 25A-25M. SnTox5 assay in Snn5 targeted M₀ wheat regenerants. (FIG. 25A) Fielder control grown from seed. (FIGS. 25B-M) independent M₀ regenerants with (FIG. 25B) homozygous wildtype; (FIGS. 25C-D) heterozygous in-frame mutant: (FIG. 25C) −3; (FIG. 25D) −6; (FIGS. 25E-H) heterozygous mutant: (FIG. 25E) −5; (FIG. 25F) +20; (FIG. 25G) +2−1; (FIG. 25H) −4; (FIGS. 25I-M) biallelic or homozygous mutant: (FIG. 25I) −11, −4; (FIG. 25J) −8, −2; (FIG. 25K) −10, −10; (FIG. 25L) +1, −2; (FIG. 25M) −5, −1 genotypes. Mutation notation is as follows: a positive number, +, indicates the number of bases inserted, a negative number, −, indicates the number of bases deleted.

FIG. 26. gRNA target sequences. Sequences from top to bottom in FIG. 26 are SEQ ID NOs:227-231.

FIG. 27. Primers used to amplify the target region for amplicon next generation sequencing. Nucleotides shown in capital letters are the 5′-stub compatible with Illumina™ NGS library preparation. Sequences from top to bottom in the left column of FIG. 15 (F Primer) are SEQ ID NOs: 232-235. Sequences from top to bottom in the right column of FIG. 15 (R Primer) are SEQ ID NOs: 236-239.

FIGS. 28A-28B. Spotting of small drops of microparticle suspension over the microcarrier disk. Multiple small drops of microparticle suspension coated with a protein or RNPs are spread over the inner circle of a microcarrier disk (FIG. 28A) Microparticles are bombarded using a helium pressured particle gun (FIG. 28B).

### DETAILED DESCRIPTION

The present disclosure relates to delivery of protein to plant cells by biolistics. Previous methods coated microparticles with protein(s), and then a suspension, containing the microparticles, is spread over the inner circle of a macrocarrier disk. The loaded macrocarrier disk is frozen in liquid nitrogen and lyophilized using a lyophilizer or air dried. However, microparticle suspensions are difficult to spread evenly and even lyophilization or air-drying can be hard to achieve due to the surface tension of the suspension.

The present disclosure describes a rapid air-drying and efficient spreading method for microparticle suspension with minimal surface tension that results in an efficient delivery of proteins to target plant cells. In addition, the present disclosure describes an efficient gene editing method in plants with high temperature treatments.

In one aspect, provided herein is a method of biolistically delivering a substance to plant tissue comprising:
(a) applying a rupture or bombardment pressure of about 100 psi to about 3000 psi to a macrocarrier disk carrying at least one drop of coated microparticles in order to bombard the coated microparticles into the plant tissue to form bombarded plant tissue, wherein the microparticles are about 0.1 μm to about 2.0 μm in size and wherein the microparticles are in a volume of microparticle suspension of about 0.1 μl to about 50 μl; and
(b) culturing the bombarded plant tissue on growth media in optimal incubation conditions for each substance.

One aspect of the invention includes a method of delivering a substance to plant tissue comprising:
(a) biolistically delivering microparticles coated with the substance to the plant tissue to generate bombarded plant tissue,
wherein the microparticles are biolistically delivered with a gene gun,
wherein 2 or more drops of microparticle suspension are spread over the inner circle of the macrocarrier disk before drying or lyophilization,
wherein a drop of microparticle suspension is in a volume of about 0.1 μl to about 10 μl,
wherein the drops of microparticle suspension are in a volume of about 0.1 μl to about 50 μl, about 3000 psi, and
(b) culturing the bombarded plant tissue on growth media in optimal incubation conditions for each substance.

In certain aspects, different methods are used to accelerate the particles: these include pneumatic devices; instruments utilizing a mechanical impulse or macroprojectile; centripetal, magnetic or electrostatic forces; spray or vaccination guns; and apparatus based on acceleration by shock wave, such as electric discharge. In certain aspects, a gene gun is used, such as a Bio-Rad PDS 1000 gun. In other aspects, a generic particle inflow gun (PIG) is used. Both of these gene guns use helium pressure.

Aspects of the invention also include a method of delivering RNPs to plant tissue comprising:
(a) biolistically delivering microparticles coated with the substance to the plant tissue to generate bombarded plant tissue,
wherein the microparticles are biolistically delivered with a gene gun,
(b) the incubating of the bombarded plant tissue is performed at about 26° C. to about 40° C., and
(c) culturing the bombarded plant tissue on growth media.

In certain aspects, the plant tissue is in an immature embryo (IE), callus tissue, somatic embryo, zygotic embryo, meristem tissue, pollen, cotyledon, leaf, stem, or root tissue.

In certain aspects, the plant is a crop plant.

In certain aspects, the crop plant is wheat, rice, maize, soybean, pepper, alfalfa, sunflower, cassava, cacao, banana, potato, sweet potato, strawberry, grape, poplar, coffee, walnut, almond, peach, nectarine, plum, apricot, apple, pear, persimmon, blueberry, blackberry, raspberry, or a fruit tree.

In certain aspects, the microparticles are about 0.1 μm to about 2.0 μm in size.

In certain aspects, the microparticles are about 0.4 to about 1.6 μm in size

In certain aspects, the microparticles are about 0.5 μm to about 0.7 μm in size

In certain aspects, the microparticle is about 0.6 μm in size.

In certain aspects, the rupture pressure is about 100 psi to about 3000 psi.

In certain aspects, the rupture pressure is about 500 psi to about 2300 psi.

In certain aspects, the rupture pressure is about 600 psi to about 2200 psi.

In certain aspects, the rupture pressure is about 650 psi to about 1350 psi.

In certain aspects, the rupture pressure is about 1100 psi.

In certain aspects, the incubating of the bombarded plant tissue is performed at about 26° C. to about 40° C.

In certain aspects, the incubating of the bombarded plant tissue is performed at about 30° C. to about 37° C.

In certain aspects, the incubating of the bombarded plant tissue is performed at about 34° C.

In certain aspects, the incubating of the bombarded plant tissue is performed for about 1 hour to about 180 days post-bombardment.

In certain aspects, the incubating of the bombarded plant tissue is performed for about 24 hours to about 30 days post-bombardment.

In certain aspects, the incubating of the bombarded plant tissue is performed for about 2 days to about 14 days post-bombardment.

In certain aspects, the incubating of the bombarded plant tissue is performed for about 7 days post-bombardment.

In certain aspects, the incubating of the bombarded plant tissue is performed for about 3 days post-bombardment.

In certain aspects, the substance on the microparticle comprises a protein of interest.

In certain aspects, the substance on the microparticle comprises Cas9 protein.

In certain aspects, the substance on the microparticle comprises nucleic acid.

In certain aspects, the substance on the microparticle comprises sgRNA-Cas9 ribonucleoproteins (RNPs).

In certain aspects, the microparticle suspension is in a volume of about 1 μl to about 50 μl per bombardment. In certain aspects, the microparticle is in a volume of about 10 μl to about 30 μl per bombardment. In certain embodiments, the microparticle suspension is in a volume of 1 μl, 2 μl, 3 μl, 4 μl, 5 μl, 6 μl, 7 μl, 8 μl, 9 μl, 10 μl, 11 μl, 12 μl, 13 μl, 14 μl, 15 μl, 16 μl, 17 μl, 18 μl, 19 μl, 20 μl, 21 μl, 22 μl, 2 μl 3, 24 μl, 25 μl, 26 μl, 27 μl, 28 μl, 29 μl, 30 μl, 31 μl, 32 μl, 33 μl, 34 μl, 35 μl, 36 μl, 37 μl, 38 μl, 39 μl, 40 μl, 41 μl, 42 μl, 43 μl, 44 μl, 45 μl, 46 μl, 47 μl, 48 μl, 49 μl, or 50 μl. In certain aspects, the suspension is applied as one or more drops, and then is spread over the inner disc of thee microcarrier using a spatula, a pipette tip or other mechanical means.

### Bombardment of Plant Tissue

DNA-free genome editing technology using CRISPR/Cas9 ribonucleoprotein complexes (RNPs) has recently become a valuable tool, especially in crop species that are vegetatively propagated because transgenes do not get segregated away as is seen in standard plant breeding processes. Targeted mutagenesis has been demonstrated after the direct delivery of preassembled Cas9-RNPs in various plant protoplast systems. Some have produced edited plants following the transfection of single protoplasts; however, regeneration of several crop plant protoplasts is not feasible and genotype-dependent with current methods. Biolistic delivery of Cas9-RNPs into maize, wheat and rice embryos has been also reported, but editing rates have generally been low or sometimes not reproducible.

In the method described herein, drops of microparticle suspensions are deposited and dried on a macrocarrier disk. The microparticles used in the method have been coated with RNPs comprising the Cas9 protein complexed with guide and tracer RNAs. Bombardment parameters used are selected for a specific microparticle size range, rupture disk pressure and number of shots for most efficient transformation. The plant tissues targeted for transformation may be selected from a wide range of sources including callus, somatic embryos, zygotic embryos, meristem tissue, pollen, cotyledon, leaf, stem, and root tissue.

A microcarrier (also called a “microparticle” or a “microprojectile”) is a small gold, tungsten or another microparticle used for bombardment by a gene gun. Microcarriers are coated with any reagent such as DNA, RNA, protein or RNPs before bombardment to target cells. In certain embodiments, the microcarrier (gold particle) is 0.4 μm, 0.6 μm, 1.0 μm, or 1.6 μm in size.

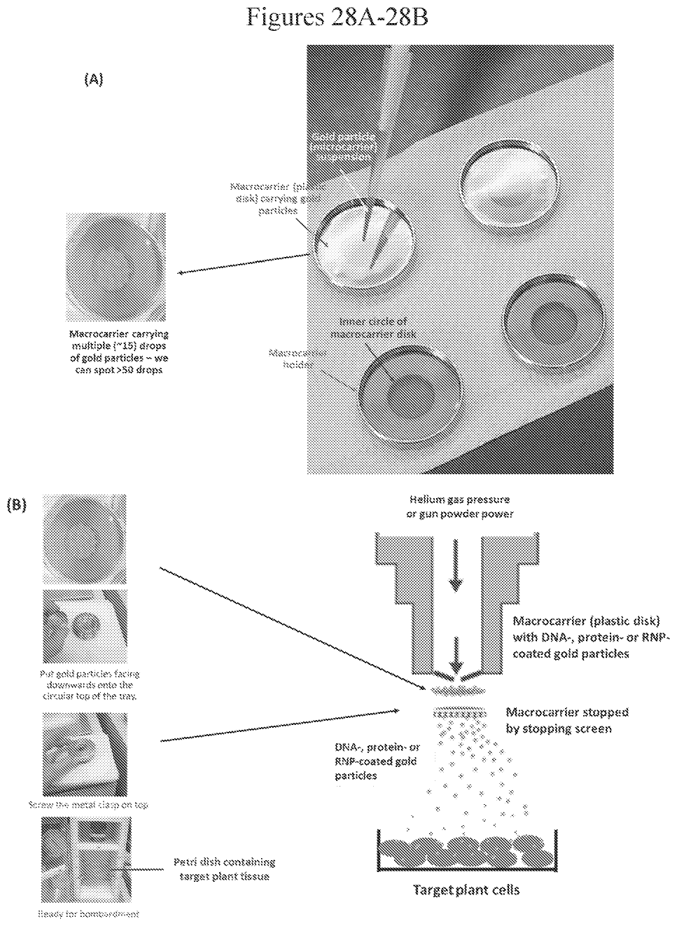

A “macrocarrier” is a solid substrate (such as a disk) on which the microcoarriers (e.g., gold microparticles) can be deposited. FIGS. 28A-28B provides an illustration of one embodiment of the present method.

### Nucleic Acids

The term “nucleic acid” refers to deoxyribonucleic acid (DNA) or ribonucleic acid (RNA) and polymers thereof in either single- or double-stranded form, composed of monomers (nucleotides) containing a sugar, phosphate and a base that is either a purine or pyrimidine.

Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. A “nucleic acid fragment” is a portion of a given nucleic acid molecule.

The terms “polynucleotide,” “nucleic acid” and “nucleic acid fragment” are used interchangeably herein. These terms encompass nucleotides connected by phosphodiester linkages. A “polynucleotide” may be a ribonucleic acid (RNA) or deoxyribonucleic acid (DNA) polymer that is single- or double-stranded, that optionally contains synthetic, non-natural or altered nucleotide bases. A polynucleotide in the form of a polymer of DNA may comprise one or more segments of cDNA, genomic DNA, synthetic DNA, or mixtures thereof. Nucleotide bases are indicated herein by a single letter code: adenine (A), guanine (G), thymine (T), cytosine (C), inosine (I) and uracil (U). Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

A “nucleotide sequence” is a polymer of DNA or RNA that can be single-stranded or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” are used interchangeably and may also be used interchangeably with gene, cDNA, DNA and RNA encoded by a gene.

The invention encompasses isolated or substantially purified nucleic acid compositions. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. Fragments and variants of the disclosed nucleotide sequences are also encompassed by the present invention. By “fragment” or “portion” is meant a full length or less than full length of the nucleotide sequence.

“Naturally occurring,” “native,” or “wild-type” is used to describe an object that can be found in nature as distinct from being artificially produced. For example, a protein or nucleotide sequence present in an organism (including a virus), which can be isolated from a source in nature and that has not been intentionally modified by a person in the laboratory, is naturally occurring.

“Genome” refers to the complete genetic material of an organism.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al. 1984), BLASTP, BLASTN, and FASTA (Altschul, S. F., et al., 1990. The BLASTX program is publicly available from NCBI and other sources (BLAST Manual, Altschul, S., et al., NCBI NLM NIH Bethesda, Md. 20894; Altschul, S., et al., 1990). The well-known Smith Waterman algorithm may also be used to determine identity.

### Promoters

“Promoter” refers to a nucleotide sequence, usually upstream (5′) to its coding sequence, that controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions. A general discussion of promoters is provided in U.S. Pat. No. 7,501,129, which is incorporated by reference herein.

The invention encompasses isolated or substantially purified nucleic acid compositions. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. Fragments and variants of the disclosed nucleotide sequences are also encompassed by the present invention. By “fragment” or “portion” is meant a full length or less than full length of the nucleotide sequence.

In certain embodiments, the present invention provides vectors and expression cassettes containing the promoters described above. A “vector” is defined to include, inter alia, any viral vector, as well as any plasmid, cosmid, phage or binary vector in double or single stranded linear or circular form that may or may not be self-transmissible or mobilizable, and that can transform prokaryotic or eukaryotic host either by integration into the cellular genome or exist extrachromosomally (e.g., autonomous replicating plasmid with an origin of replication).

Nucleic acids encoding therapeutic compositions can be engineered into a vector using standard ligation techniques, such as those described in Sambrook and Russell, Molecular Cloning: A Laboratory Manual, Cold Spring Harbor Laboratory Press Cold Spring Harbor, NY (2001). For example, ligations can be accomplished in 20 mM Tris-Cl pH 7.5, 10 mM MgCl2, 10 mM DTT, 33 μg/ml BSA, 10 mM-50 mM NaCl, and either 40 M ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 30-100 μg/ml total DNA concentrations (5-100 nM total end concentration).

In certain embodiments, the present invention provides a vector containing an expression cassette comprising a promoter operably linked to a target sequence (e.g., PEDV S1 protein) for production of vaccine. “Expression cassette” as used herein means a nucleic acid sequence capable of directing expression of a particular nucleotide sequence in an appropriate host cell, which includes a promoter operably linked to the nucleotide sequence of interest that may be operably linked to termination signals. The coding region usually codes for a functional RNA of interest, for example an RNAi molecule. The expression cassette including the nucleotide sequence of interest may be chimeric.

“Operably-linked” refers to the association of nucleic acid or amino acid sequences on single molecular fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. Nucleic acid is “operably linked” when it is placed into a functional relationship with another nucleic acid sequence. Generally, “operably linked” means that the DNA sequences being linked are contiguous. However, enhancers do not have to be contiguous. Linking is accomplished by ligation at convenient restriction sites. If such sites do not exist, the synthetic oligonucleotide adaptors or linkers are used in accordance with conventional practice. Additionally, multiple copies of the nucleic acid encoding enzymes may be linked together in the expression vector. Such multiple nucleic acids may be separated by linkers.

“Expression” refers to the transcription and/or translation of an endogenous gene or a transgene in cells. For example, in the case of antisense constructs, expression may refer to the transcription of the antisense DNA only. In addition, expression refers to the transcription and stable accumulation of sense (mRNA) or functional RNA. Expression may also refer to the production of protein.

“Expression cassette” as used herein means a DNA sequence capable of directing expression of a particular nucleotide sequence in an appropriate host cell, comprising a promoter operably linked to the nucleotide sequence of interest that is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Such expression cassettes will comprise the transcriptional initiation region linked to a nucleotide sequence of interest. Such an expression cassette may be provided with a plurality of restriction sites for insertion of the gene of interest to be under the transcriptional regulation of the regulatory regions. The expression cassette may additionally contain selectable marker genes.

### Proteins

The terms “protein,” “peptide” and “polypeptide” are used interchangeably herein.

The term “amino acid” includes the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in Dextrorotary or Levorotary stereoisomeric forms, as well as unnatural amino acids (e.g., phosphoserine, phosphothreonine, phosphotyrosine, hydroxyproline, and gamma-carboxyglutamate; hippuric acid, octahydroindole-2-carboxylic acid, statine, 1,2,3,4,-tetrahydroisoquinoline-3-carboxylic acid, penicillamine, ornithine, citruline, alpha-methyl-alanine, para-benzoylphenylalanine, phenylglycine, propargylglycine, sarcosine, and tert-butylglycine). The term also comprises natural and unnatural amino acids (Dextrorotary and Levorotary stereoisomers) bearing a conventional amino protecting group (e.g. acetyl or benzyloxycarbonyl), as well as natural and unnatural amino acids protected at the carboxy terminus (e.g., as a (C1-C₆)alkyl, phenyl or benzyl ester or amide; or as an α-methylbenzyl amide). Other suitable amino and carboxy protecting groups are known to those skilled in the art (See for example, Greene, T. W.; Wutz, P. G. M., Protecting Groups In Organic Synthesis; second edition, 1991, New York, John Wiley & sons, Inc, and documents cited therein). An amino acid can be linked to the remainder of a compound through the carboxy terminus, the amino terminus, or through any other convenient point of attachment, such as, for example, through the sulfur of cysteine.

The invention encompasses isolated or substantially purified protein compositions. In the context of the present invention, an “isolated” or “purified” polypeptide is a polypeptide that exists apart from its native environment and is therefore not a product of nature. A polypeptide may exist in a purified form or may exist in a non-native environment such as, for example, a transgenic host cell. For example, an “isolated” or “purified” protein, or biologically active portion thereof, is substantially free of other cellular material, or culture medium when produced by recombinant techniques, or substantially free of chemical precursors or other chemicals when chemically synthesized. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed proteins or partial-length proteins encoded thereby are also encompassed by the present invention. By “fragment” or “portion” is meant a full length or less than full length of the amino acid sequence of, a polypeptide or protein.

A “variant” of a molecule is a sequence that is substantially similar to the sequence of the native molecule.

“Isolated” means altered or removed from the natural state. For example, a nucleic acid or a peptide naturally present in a living animal is not “isolated.” but the same nucleic acid or peptide partially or completely separated from the coexisting materials of its natural state is “isolated.” An isolated nucleic acid or protein can exist in substantially purified form, or can exist in a non-native environment such as, for example, a host cell. Unless it is particularly specified otherwise herein, the proteins, virion complexes, antibodies and other biological molecules forming the subject matter of the present invention are isolated, or can be isolated.

The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to a reference sequence over a specified comparison window. Optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

The invention will now be illustrated by the following non-limiting Examples.

### Example 1

### Improvement of Gene Delivery and Mutation Efficiency in the CRISPR-Cas9 Wheat Genomics System Via Biolistics

### Abstract

Discovery of the CRISPR-Cas9 gene editing system revolutionized the field of plant genomics. Despite advantages in ease of designing gRNA and the low cost of the CRISPR-Cas9 system, there are still hurdles to overcome in low mutation efficiencies, specifically in hexaploid wheat. In conjunction with gene delivery and transformation frequency, the mutation rate bottleneck has the potential to slow down advancements in genomic editing of wheat. In this study, nine bombardment parameter combinations using three gold particle sizes and three rupture disk pressures were tested to establish optimal stable transformation frequencies in wheat. Utilizing the best transformation protocol and a knockout cassette of the phytoene desaturase gene, we subjected transformed embryos to four temperature treatments and compared mutation efficiencies. The use of 0.6 μm gold particles for bombardment increased transformation frequencies across all delivery pressures. A heat treatment of 34° C. for 24 hours resulted in the highest mutation efficiency with no or minimal reduction in transformation frequency. The 34° C. treatment produced two M₀ mutant events with albino phenotypes, requiring biallelic mutations in all three genomes of hexaploid wheat. Utilizing optimal transformation and heat treatment parameters greatly increases mutation efficiency and can help advance research efforts in wheat genomics.

### Introduction

Modern development of wheat varieties is obtained through indirect or direct gene transfer, typically via Agrobacterium and particle bombardment, respectively. Through Agrobacterium-mediated transformation, selected genes are transferred from bacteria to plant cells via disarmed Agrobacterium vectors and whole plants are then generated through tissue culture. Agrobacterium-mediated transformation has been well established in a variety of crops and is advantageous for intact transfer of larger DNA fragments. Although Agrobacterium-mediated transformation can result in high instances of single copy events and intact T-DNA delivery, it lacks consistency across species, genotypes and tissue types especially in more recalcitrant varieties. This specificity limits the ability for impact across multiple crops or cultivars in addition to more regulatory hurdles involved with the transfer of Agrobacterium binary backbone.

Conversely, particle bombardment is a method of direct gene transfer in which DNA is precipitated onto gold particles and delivered onto plant tissue using high pressure Helium gas. In this system there are multiple factors that can be adjusted to optimize DNA delivery such as the size of gold particles, amount of gold particles, delivery pressure, amount of DNA and distance from the plant tissue. We chose to use particle bombardment as our gene delivery system because it is less species and genotype dependent. Although particle bombardment can have higher instances of multiple copy events, the copies are often located on the same locus allowing for easy segregation out in future generations. In addition, since particle bombardment is a form of direct gene delivery it is a mechanism that is unregulated and can result in a more streamlined regulatory process.

In the forefront of genetic engineering today is gene editing and CRISPR. The CRISPR-Cas9 gene editing system is widely used for improvement of various field crops. The system allows researchers to utilize short repeats of endogenous DNA in the plant genome derived from bacteriophages to identify specific locations in the genome for gene editing. In conjunction with these DNA repeats, the Cas9 protein is programmed to cut double-stranded DNA in precise locations and allows for site-specific editing within the plant genome. The ability to make site-specific gene edits in a plant gives this technology an advantage over traditional genetic modification methods that randomly insert DNA. The CRISPR-Cas9 system allows for not only the insertion of new DNA into the plants but also the deletion or silencing of single genes within the genome that can confer a variety of advantageous phenotypes.

Mutation efficiencies using the CRISPR-Cas9 system vary widely across monocot species. Rice mutation efficiencies can exceed a 60% mutation rate, whereas wheat mutation efficiencies remain at about 10% or less. However, mutation efficiencies are largely impacted by the individual components of a construct. The proper selection of promoters to drive expression of Cas9 and sgRNAs can increase expression levels and ultimately positively impact mutation efficiency in transformed plants. In addition, testing sgRNA sequence efficiency in vivo before stable transformation is critical for maximizing mutation efficiency. Even the sequence of Cas9 protein can affect mutation efficiency, through codon optimization and the presence or absence of introns. At the plant level, mutation efficiency is dependent on establishing an effective tissue culture protocol which is predominantly reliant on identifying a good starting explant. Another approach to increase mutation rate in plants are the effects of temperature treatments. The effect of temperature treatments on mutation efficiencies has been proven in mammalian cell culture. The CRISPR-Cas9 system was established on a principle derived from Streptococcus pyogenes adaptive immunity to viruses. S. pyogenes grows the most dynamically at 40° C. It is reasonable to expect that the Cas9 protein will be more efficient at higher temperatures. Recent studies have reported positive effects of temperature treatment on gRNA activity in plants.

The phytoene desaturase gene (PDS) is commonly applied as a demonstration of experimental mutation rates due to its visual phenotype and wide conservation across species. The PDS gene is involved in the carotenoid synthesis pathway in plants. A recessive mutation, or knockout, of the PDS gene disrupts the formation of beta-carotene and confers a visual albino phenotype. The PDS knockout has been demonstrated in a range of species including, but not limited to, Arabidopsis, rice, banana, cassava and melon. The PDS knockout, albino phenotype, has not been previously reported in M. hexaploid wheat because it is likely to require biallelic mutations on all six loci of the three genomes.

Finding a good combination of these factors to increase CRISPR mutation efficiency in wheat is a valuable tool for facilitating the production of robust wheat cultivars that can withstand the effects of climate change faster than other approaches. In this study, we establish parameters for particle bombardment that result in improved transformation frequencies, as well as subsequent temperature treatments to increase mutation efficiencies in hexaploid wheat. We also report the successful generation of PDS triple recessive mutant events in the M₀ generation displaying the albino phenotype. In addition, we demonstrate the albino phenotype in M₁ progeny plants derived from an edited event with monoallelic and biallelic mutations in the three different genomes.

### Materials and Methods

### Plant Material

Seeds of Triticum aestivum L. cv. Fielder were sown weekly and grown in growth chambers under 16-hr days at 24° C., and 8-hr nights at 15° C. Light levels were set to approximately 130 μmol m⁻² s⁻¹ at head height. Immature spikes were harvested 10-14 days post flowering with an immature embryo (IE) sized 1.7-2.2 mm. Immature spikes were collected up to 5 days pre-bombardment and stored at 4-6° C. One day prior to bombardment, immature seeds were harvested from immature spikes and surface-sterilized using 20% (v/v) bleach (8.25% sodium hypochlorite) plus one drop of Tween 20 for 15 minutes before triple rinsing with sterile water. IEs were then isolated and placed scutellum side up on DBC3 medium and incubated at 26° C. overnight.

### Plasmids

Plasmids, pAct1IH-PT-4, pAct1IDsRED and pRGE610-PDS-PS2, were used for transformation (FIGS. 1A-1C). pAct1IIPT4 and pAct1IDsRED contain hygromycin phosphotransferase (hpt) and DsRED genes, respectively, each under control of the rice actin 1 promoters, its intron (act1I) and the nos 3′ terminator. pRGE610-PDS-PS2 contains Cas9 gene and gRNA cassettes and was made using the following steps. First, the wheat U6 promoter with blue-white screening cassette was amplified from pTaU6-sgRNA (gift from Daniel Voytas) with primers TaU6Lac-HindIII-F (5′-TAAAGGAACCAATTCAGTCGACTGGAT-3′ SEQ ID NO:1) and TaU6Lac-SbfI-R (5′-GCCCTGCAGGTCTAGATATCTCGAGGGTACCAAACTGAG-3′ SEQ ID NO:2). pRGE32 (gift from Yinong Yang, Addgene ID 63159) was digested with SbfI and HindIII to release the original sgRNA cassette. Then, the PCR fragment from the first step was ligated into the digested pRGE32 backbone to create pRGE610. Next, two fragments from pGTR (gift from Yinong Yang, Addgene ID 63143) were amplified. The first fragment, product of TaU6-L5AD-BtgZI-F (5′-CGGGTCTCACTTGGCGATGTCTTGGTCTGCTTGACAAAGCACCAGTGG-3′ SEQ ID NO:3) and PDS-PS2-gR (5′-TAGGTCTCAAGGTGGTCATTGCACCAGCCGGG-3′ SEQ ID NO:4), contained a tRNA and the first half of the sgRNA spacer PS2 with 4 bp overhangs on each site to enable Golden Gate assembly with the second fragment. The second fragment, product of PDS-PS2-gF (5′-CGGGTCTCCACCTTCTTTTCAGCGTTTTAGAGCTAGAA-3′ SEQ ID NO:5) and L3AD-BtgZI-R (5′-TAGGTCTCCAAACGCGATGGAGCGACAGCAAACAAAAAAAAAAGCACCGACTCG-3′ SEQ ID NO:6), contained the second half of PS2 followed by the sgRNA scaffold and overhangs for Golden Gate assembly on each site. The NEB® Golden Gate Assembly Kit (BsaI-HF®v2) was used to combine both fragments. The resulting product was used as template for primers TaU6-S5AD-BtgZI-F (5′-CGGGTCTCACTTGGCGATGTCTTGGTCTGCTTG-3′ SEQ ID NO:7) and S3AD-BtgZI-R (5′-TAGGTCTCCAAACGCGATGGAGCGACAGCAAAC-3′ SEQ ID NO:8) to yield enough of the Golden Gate assembly product for digestion with BtgZI. The BtgZI digested product was then ligated into the BsaI digested backbone of pRGE610, to finally create pRGE610-PDS-PS2.

### Stable Transformation Via Particle Bombardment

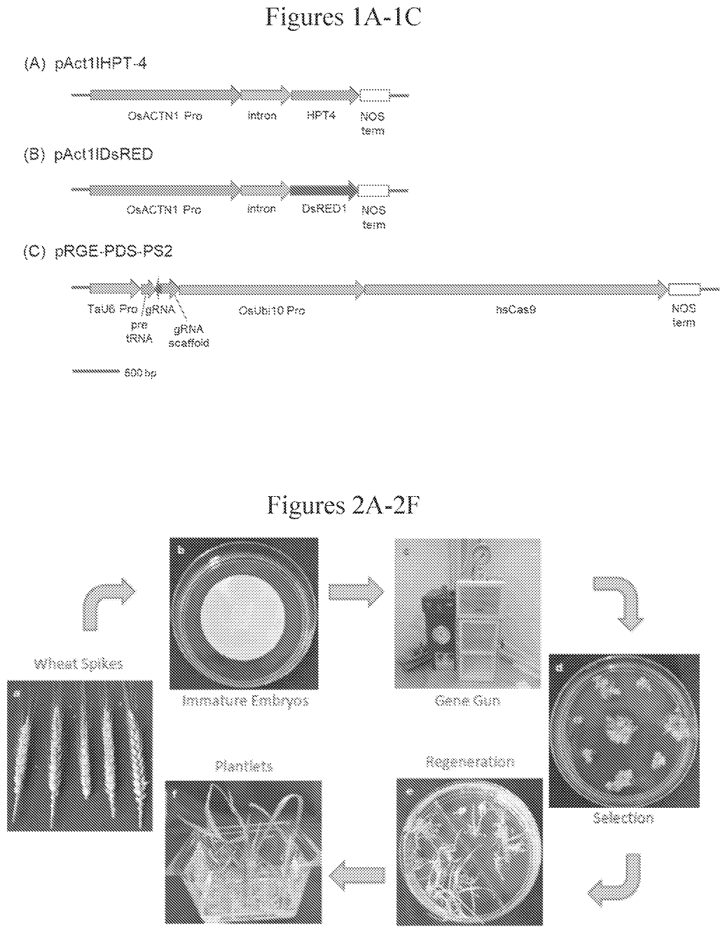

IEs isolated from immature spikes (FIG. 2A) and pre-incubated at 26° C. overnight were used for bombardment. On the day of bombardment, IEs were placed on top of a 40 mm filter paper on a plate of DBC3 osmoticum medium containing mannitol and sorbitol (0.2 M each) (FIG. 2B). Four hours after treatment with osmoticum, IEs were bombarded using Bio-Rad PDS-1000/He particle gun (FIG. 2C). Two milligrams of gold particles (0.4, 0.6 and 1.0 μm) were coated with 5 μg of a mixture of pAct1IHPT4 and pActIIDsRED or pAct1IHPT4 and pRGE610-PDS-PS2 at a 1:2 ratio. Each particle prep was resuspended in 85 μL of 100% EtOH and 7.5 μL was spread onto the center of a macrocarrier inside of a macrocarrier holder. The particle preps were used for bombardment with a Bio-Rad PDS-1000/He biolistic device (Bio-Rad, Hercules, CA) at 3 different delivery pressures (650, 900 and 1100 psi). Each plate of IEs was bombarded twice per treatment. After bombardment, IEs were transferred from filter paper to the exposed media and incubated overnight at 26° C. in dim light (3-10 μmol m² s⁻¹). Sixteen hr post bombardment, IEs were transferred to DBC3 medium and incubated at 26° C. for 1 wk in dim light. Following the resting period, IEs went through 3 rounds of selection via DBC3 media containing 30 mg/l hygromycin B, each round of selection lasting 3 wk (FIG. 2D). After the third round of selection, regeneration was initiated using DBC6 media (Cho et al. 2015) containing 30 mg/l hygromycin B and incubated at 26° C. in high light (90 μmol m² s⁻¹) and subcultured every 3 wk. Once shoots were approximately 0.5 cm in height (FIG. 2E), shoots were transferred to WR rooting media for root formation. Plantlets were then transferred to soil once they had enough shoots to support transplant to soil (FIG. 2F).

### Temperature Treatment of Bombarded Immature Embryos

Bombarded IEs were subjected to varying heat treatments of 26° C., 30° C., 34° C. and 37° C. 4 days post bombardment for 24 hr, incubated in Heratherm impact Microbiological Incubators (Cat #50125590, Thermo Scientific, Grand Island, NY). Non-bombarded IEs with temperature treatments were tested for tissue culture response. The IEs were isolated onto DBC3 medium and incubated for 1 day. The following day they were placed on DBC3 osmoticum medium for 20 hours to replicate the same treatment as bombarded IEs. The IEs were then transferred back to DBC3 medium for 3 days and then split up and treated with heat treatments of 26° C., 30° C., 34° C. and 37° C. for 24 hours. IEs were allowed to grow and proliferate into callus for 5 weeks at which point the callus in each plate was collectively weighed for comparison.

### Fluorescent Visualization

Fluorescent images of IEs, calla and plantlets of transgenic Fielder events were visualized with a fluorescent Leica M165 FC stereomicroscope, equipped with Leica DFC7000 T (JH Technologies, Fremont, CA), using two microscopic filters, brightfield and ET DSR with 545 nm excitation and 620 nm emission. The microscope is linked to a camera imaging software, Leica Application Suite version 4.9, which was used to capture the fluorescent images. Screening of fluorescent activity was measured at different magnifications.

### Detection of Transgenes and CRISPR/Cas9 Mutations

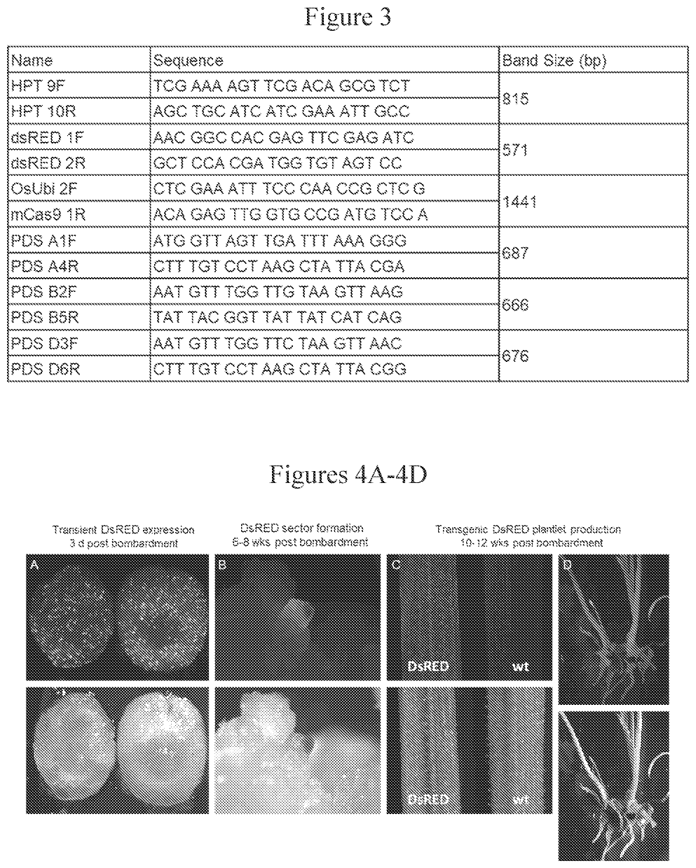

Genomic DNA was extracted from leaf tissue following a CTAB extraction method. The dsRED, hpt and Cas9 transgenes were confirmed by PCR using sequence specific primers (FIG. 3). Amplifications were performed in a 25-μl reaction with DreamTaq PCR Master Mix (2×) (Thermo Fisher Scientific, Grand Island, NY). For each PCR reaction, 23 μL were loaded onto a 0.8% agarose gel for electrophoresis.

For detection of PDS mutations, a fragment within range of the desired mutation was targeted via homoallele specific primers across each genome (FIG. 3 and amplified by PCR. The amplified PCR product was cut and DNA was extracted from gel samples using the Qiagen QIAquick® Gel Extraction Kit (QIAGEN, Chatsworth, CA). The purified DNA samples were used for Sanger sequencing. Mutation efficiency was calculated as the number of mutated unique events per treatment divided by the total number of events regenerated from the same treatment.

### M1 Mutation Screening

Heterozygous mutation line PC14A containing monoallelic mutations in the A and D genomes and a heterozygous biallelic mutation in the B genome was chosen for next generation progeny screening. M₁ IEs sized 2.0-3.0 mm were harvested off of the M₀ plant. IEs were surface sterilized with 20% bleach plus one drop of Tween 20 for 15 minutes. Sterilized IEs were triple rinsed with sterile water to remove excess bleach. Immature embryos were then excised and placed on WR medium scutellum side down for germination. Once plantlets germinated, progeny were visually assessed for phenotype and sampled for genotyping and mutation analysis.

### Results and Discussion

### Optimization of Particle Bombardment Parameters for Plant Transformation

### Gold Particle Size and Transformation Frequency

Transient DsRED expression driven by the rice actin 1 promoter and its intron was initially detected 1 day after particle bombardment and was clear 2 to 3 days post bombardment (FIG. 4A). DsRED-expressing sectors were formed (FIG. 4B), and stably transformed plantlets were generated 6-8 weeks and 10-12 weeks post bombardment, respectively (FIGS. 4C and D).

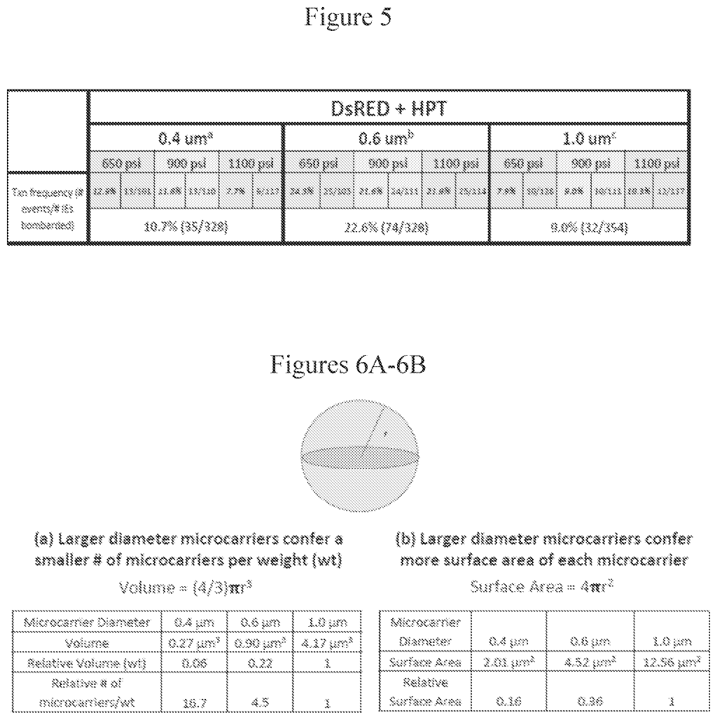

Three gold particle sizes, 0.4 μm, 0.6 μm and 1.0 μm, were tested to compare their effects on transformation frequency. The size and weight of the individual particles has an effect on its ability to physically deliver DNA to the plant cell. It was observed that the 0.6 μm gold particles performed the best in transformation frequency across all delivery pressures with an average frequency of 22.6% (FIG. 5).

The 0.4 μm particles performed second best but with an average transformation frequency of 10.7% that measured well below that of 0.6 μm. However, the 1.0 μm particles resulted in the lowest average transformation frequency of 9.0%. In our study, we used the same weight of gold particles and the same amount of DNA per prep, meaning that the larger-sized particles will have fewer particles than the smaller-sized ones. Theoretically 1.0 μm particles have 4.5-fold and 16.7-fold fewer number of particles per weight than 0.6 μm and 0.4 μm particles, respectively, because gold particle volume (weight) is calculated as (4/3)πr³ (r=radius) (FIG. 6A). Therefore, the use of 1.0 μm gold particles resulted in a lower transformation frequency likely due to a smaller number of particles per bombardment compared to 0.6 μm particles (FIG. 5; FIG. 6A). In addition, the large particles can damage the cells beyond their ability to recover and subsequently negatively affects regeneration of transgenic plants. However, our results from the 0.4 μm vs 0.6 μm comparison showed that transformation frequency (22.6%) with 0.6 μm particles were 2.1-fold higher than that (10.7%) with 0.4 μm particles (FIG. 5), even though the number of 0.6 μm particles were 3.7-fold less than that of 0.4 μm particles (FIG. 6A). This is possibly due to the smaller amount of DNA coated onto 0.4 μm particles or reduced capability of 0.4 μm particles to penetrate the target cells of IEs, compared to 0.6 μm particles.

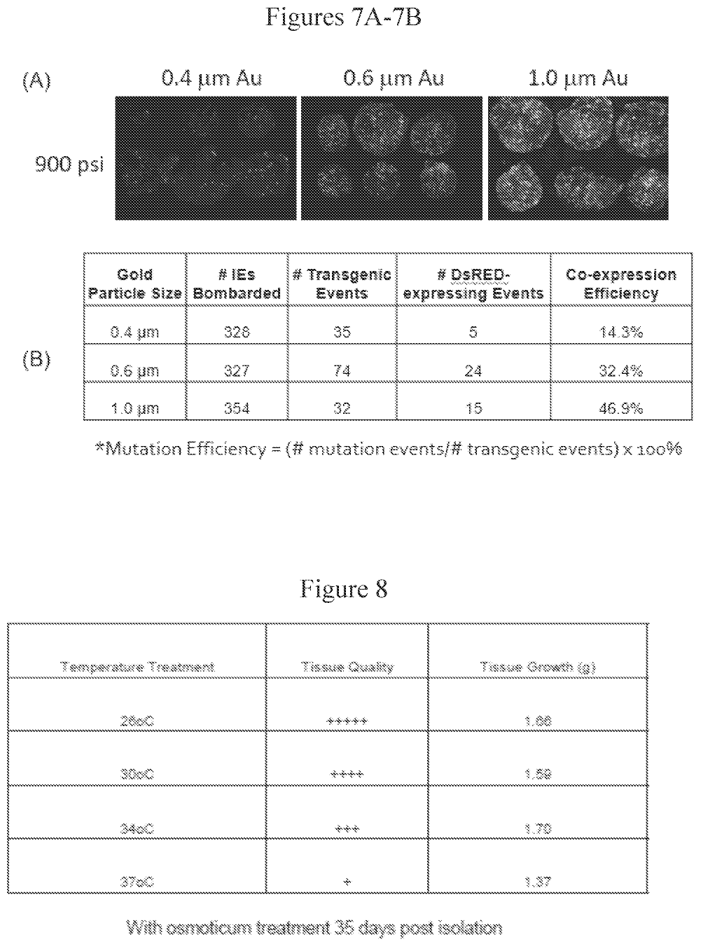

The theoretical calculation of the surface area of a sphere (4πr²) shows us that gold particle size positively correlates to surface area (FIG. 6B). As the diameter, or radius, of a gold particle increases so does the surface area of the sphere. This means that particle size alters the DNA-holding capacity of a single particle. From 0.4 μm to 1.0 μm diameters, the surface area increases by 6.25-fold. In our study, we used the same weight of gold particles and the same amount of DNA per prep, meaning theoretically the 1.0 μm gold particles can hold 6.25-fold more DNA than the 0.4 μm particles allowing for a higher percentage of DNA delivery upon impact with the plant cells. Our data supports this theory, indicating that co-expression efficiency increases as particle diameter increases (FIG. 7B) even though transient DsRED expression was weaker with smaller sizes of particles (FIG. 7A). Co-expression efficiency is calculated as the number of events visually expressing dsRED over the total number of events generated. The 1.0 μm particle size had the highest co-expression efficiency of 46.9%, 0.6 μm in the middle with 32.4% and 0.4 μm was the lowest with a co-expression efficiency of 14.3% (FIG. 7B). However, when optimizing transformation, 1.0 μm was not selected as a candidate due to its low transformation frequency regardless of its high DNA delivery performance.

### Delivery Rupture Pressure and Transformation Efficiency

Different species and tissue types can require different rupture pressures to optimize transformation frequency. We tested three rupture pressures, 650 psi, 900 psi and 1100 psi for each of the three particle sizes (FIG. 5). For the 0.4 μm particle size, both the 650 psi and 900 psi resulted in higher transformation frequencies of 12.9% and 13.6%, respectively, compared to 1100 psi (7.7%). Rupture pressures of 650 psi, 900 psi and 1100 psi resulted in similar frequencies for the 0.6 μm particle size at 24.3%, 21.6% and 21.9%, respectively. The 1.0 μm particle size resulted in the lowest transformation frequencies of 7.9%, 9.0% and 10.3% for the 650 psi, 900 psi and 1100 psi rupture pressures. In analyzing this data, we found that a 650 psi rupture pressure was optimal for both the 0.4 μm and 0.6 μm particle sizes while 1100 psi was optimal for the 1.0 μm particle size. However, given the low transformation frequencies, 1.0 μm at any rupture pressure is not recommended.

Initially, we hypothesized that the smaller the particle size, the higher the rupture pressure would need to be in order to maximize delivery of the particles to the tissue. However, we found that even using the smallest particle size, 0.4 μm, at the lowest pressure of 650 psi was optimal. The 1.0 μm particle size is larger and heavier and performs better at 1100 psi leaving us to conclude that a higher pressure is required to deliver larger particles to the plant tissue, indicating that larger-sized gold particles may have more resistance for penetrating the plant cells.

### Optimization of Mutation Efficiency in PDS Gene-Edited Wheat

### High Temperature Treatments and Mutation Efficiency

Although the Cas9 protein may be most active and efficient at 40° C., the plant cells cannot survive a prolonged exposure to such a high temperature. The key to finding the optimal temperature is one that satisfies both protein and plant. We initiated heat treatment tissue culture experiments testing callus growth of 10 Fielder embryos on standard DBC3 media after DBC3 osmoticum treatment with a temperature range of 26° C., 30° C., 34° C. and 37° C. for 1 day to monitor tissue morphology over time. To quantify the effect of heat treatment on the callus tissue, we weighed the tissue 35 days post isolation. Evaluation of the callus tissue weight from each treatment allowed us to quantify the effects of heat treatment. Both the 30° C. and 34° C. heat treatments weighed similar to the control without heat treatments at 1.59 g and 1.70 g, respectively, while the control of 26° C. weighed 1.66 g (FIG. 8). This demonstrated that plant cells are capable of long-term normal to accelerated growth after subjection to a slightly increased temperature for a short period of time. The 37° C. plate, however, grew at a slower rate, weighing in at only 1.37 g. This tells us that higher temperatures even for short periods of time negatively affects tissue growth over time. Negative effects on the growth rate will impact transformation frequencies and thus mutation efficiencies of experiments.

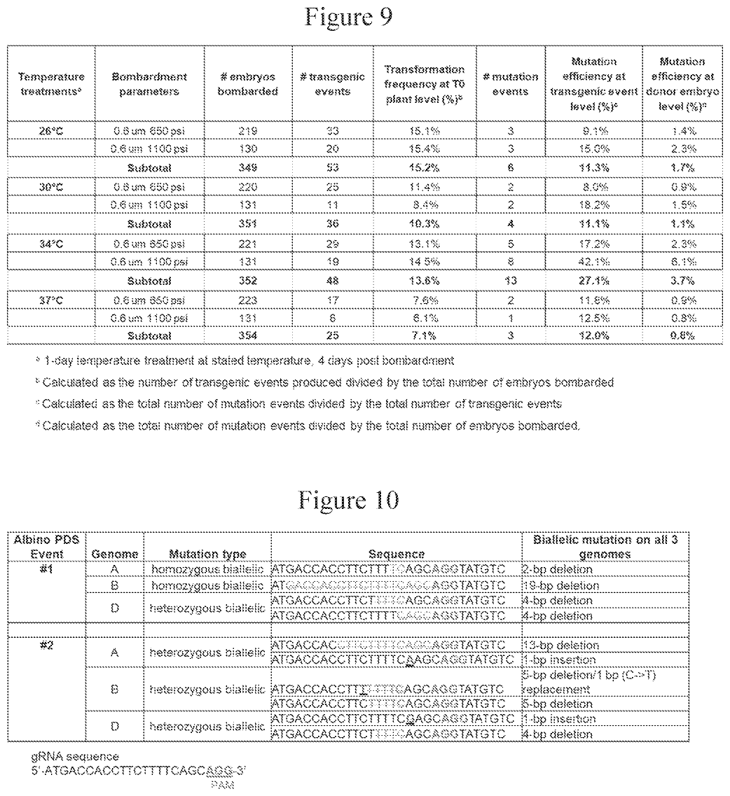

We designed our PDS mutation efficiency experiment to confirm the effects of heat treatment on mutation efficiency in plants on a significant scale side by side with our dsRED+HPT transformation frequency experiment. We chose our bombardment parameters based on the data set with the most promising transient dsRED expression and transformation efficiency. We used 0.6 μm at 2 different rupture pressures, 650 psi and 1100 psi and tested a total of 4 temperature treatments 26° C., 30° C., 34° C. and 37° C. for 24 hours, 4 days post bombardment (FIG. 9). We expected to see higher mutation frequencies at higher temperatures because of previously reported increased Cas9 activity at higher temperatures consistent with previous studies testing 22° C., 28° C., 32° C. and 37° C. in rice protoplasts, maize plants and Arabidopsis. In the earlier study, temperatures between 28° C. and 32° C. proved to increase the Cas9 activity in vivo. However, their mutation efficiencies were recorded via production of M₁ mutants via heat treatment of M₀ maize plants containing Cas9 and gRNA; transgenic plantlets were not produced via transformation in rice. We added 34° C. as a treatment because it is the temperature at which harvested seed is treated pre-germination. We believed it would be the highest temperature that does not negatively affect tissue morphology and survival, allowing plant cells to regenerate full M₀ plantlets while the Cas9 protein is able to function at a temperature more closely aligned with its bacterial origin allowing for higher protein activity. In this study, we found that regardless of bombardment parameters, a 34° C. heat treatment has the most drastic positive effect on mutation frequency up to 2.8-fold higher than any other temperature (FIG. 9). Heat treatment of 34° C. for both rupture pressures, 650 psi and 1100 psi with a particle size of 0.6 μm the mutation frequency at the transgenic event levels were 17.2% and 42.1% respectively. All other heat treatments for the same bombardment parameters were comparable. For the 650 psi and 1100 psi rupture pressure, mutation efficiencies measured 9.1% and 15.0% for the 26° C. treatment, 8% and 1⁸0.2% for the 30° C. temperature and finally 11.8% and 1²0.5% for the 37° C. temperature, respectively. There was a trend of having higher mutation efficiencies at 1100 psi than 650 psi (FIG. 9). As a result of this data, we can conclude that 34° C. is the optimal temperature at which both the plant and overexpressed Cas9/gRNA can operate to achieve the highest mutation efficiency.

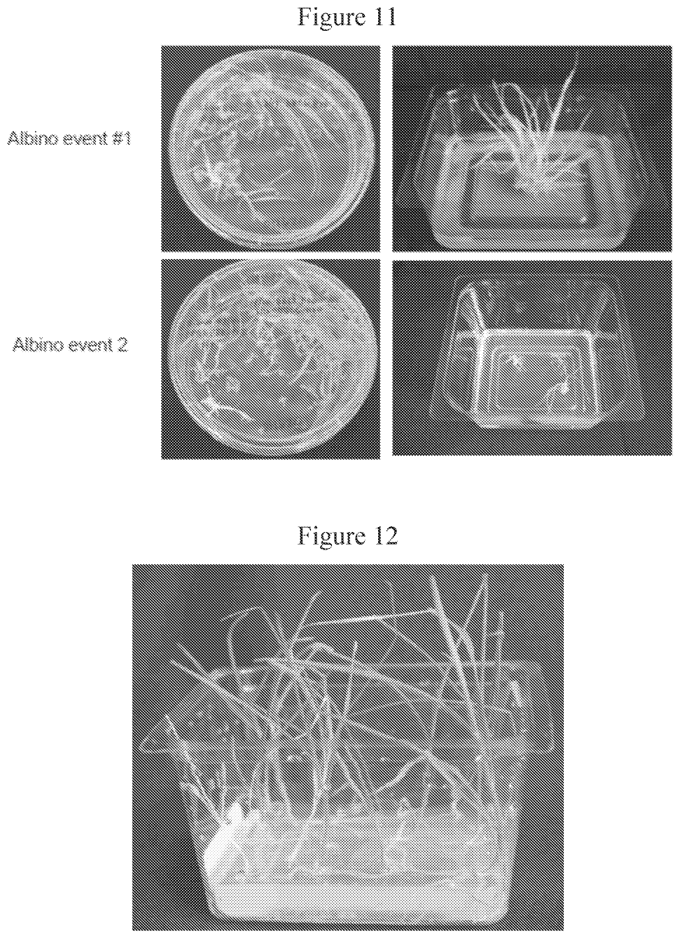

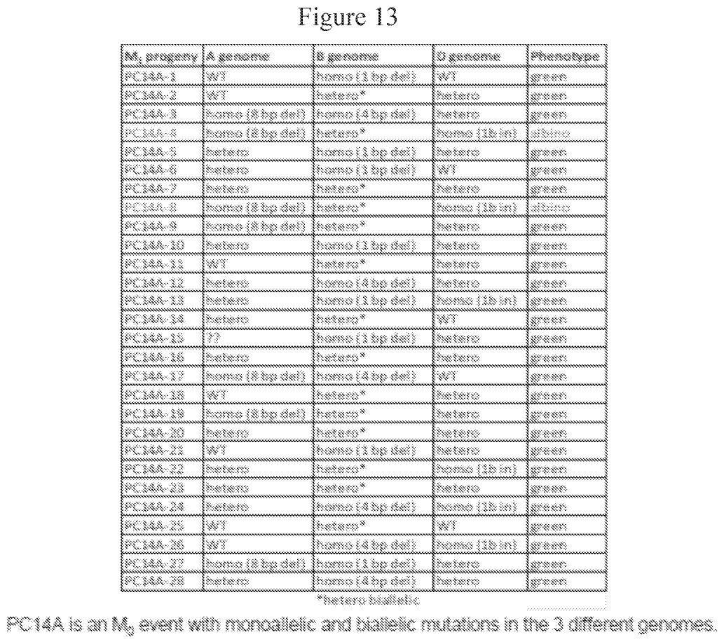

Through the use of heat treatments, we were able to obtain a variety of M₀ mutant genotypes within singular genomes as well as across multiple genomes. In the M₀ generation, 79.3% ( 23/29) of the mutants produced were single genome mutations, 6.9% ( 2/29) were two genome mutations and 13.8% ( 4/29) were triple mutants. Of the four triple-mutants, two M₀ events contain biallelic mutations across all three genomes resulting in the PDS knockout albino phenotype; all mutations were out of frame. (FIG. 10; FIG. 11). In order to obtain the albino PDS phenotype, biallelic mutations in all three genomes or mutations on all 6 loci are required. To our knowledge, this is the first report of generating wheat plants with the albino PDS phenotype at the M₀ level. Previous studies have achieved M₀ triple-mutant knockouts but were not able to achieve a phenotype in the M₀ generation. Triple-mutation knockouts were generated on the TaQsd1 gene for inhibition of preharvest sprouting in the M₁ generation by crossing a M₀ triple-mutant consisting of two biallelic mutations and one monoallelic mutation to WT fielder and segregating in future generations. Both of our M₀ biallelic triple-mutants were derived from the 34° C. heat treatment, supporting the hypothesis that heat treating transformed material increases the activity of the Cas9 protein/gRNA resulting in higher mutation efficiencies. In order to demonstrate the albino phenotype in M₁ progeny plants derived from a PDS gene-edited event, we used event PC14A having monoallelic mutations on both A and D genomes and a heterozygous biallelic mutation on the B genome. M₁ progeny from 2 out of 28 germinated seedlings of PC14A demonstrated the albino phenotype and showed a 15:1 segregation pattern (FIG. 12; FIG. 13). Genotyping analysis of these 2 albino phenotype events resulted in homozygous biallelic mutations on both A and D genomes and a heterozygous biallelic mutation on B genome (FIG. 13) confirming that the albino phenotype requires biallelic mutations across all three genomes.

In conclusion, we improved transformation efficiencies in Fielder across all delivery pressures using 0.6 μm gold particles for bombardment. We also successfully demonstrated an increase in mutation efficiency using heat treatments post bombardment. A heat treatment of 34° C. for 24 hours post bombardment resulted in the highest mutation frequency and derived a mutant albino phenotype in the M₀ generation which requires biallelic mutations in all three genomes of hexaploid wheat. Utilizing optimal transformation parameters and a 34° C. heat treatment greatly increases mutation efficiency in hexaploid wheat and can help advance research efforts in wheat genomics. The results in this study can be applied to optimize the transformation frequency and improve mutation efficiency in other crop species.

### Example 2

### Effect of Number of Drops of Protein-Coated Gold Microparticle Suspension Per Volume on Drying Time and Macrocarrier-Covering Area

### Plant Materials and Production of Embryogenic Callus Tissues

Seeds of Triticum aestivum L. cv. Fielder were sown weekly and grown in growth chambers under 16-hr days at 24° C., and 8-hr nights at 15° C. Light levels were set to approximately 130 μmol m⁻² s⁻¹ at head height. Immature spikes were harvested 10-14 days post flowering with an immature embryo (IE) sized 1.7-2.2 mm. Immature spikes were collected up to 5 days pre bombardment and stored at 4-6° C. One day prior to bombardment, immature seeds were harvested from immature spikes and surface-sterilized using 20% (v/v) bleach (8.25% sodium hypochlorite) plus one drop of Tween 20 for 15 minutes before triple rinsing with sterile water. IEs were then isolated and placed scutellum side up on DBC3 medium (Cho et al. 1998) and incubated at 26° C. overnight.

### Gold Particle Preparation for Bombardment

Purified DsRED protein were precipitated onto 0.6 μm gold particles (Bio-Rad, Hercules, CA) with the cationic lipid polymer, TransIT-2020 (Mirus, Madison, WI. Briefly, for 4 shots, 80 μl of purified DsRED protein at 0.1 μg/μl was mixed gently with 30 μl sterile gold particles (10 μg μl⁻¹ water suspension) and 2.0 μl TransIT-2020. The DsRED gold particles were pelleted in a mini microcentrifuge at 2,000 g for 30 s. The supernatant was removed, and the gold particles were resuspended in 40 μl of sterile water by brief sonication. The coated gold particles were immediately applied to 4 macrocarriers (10 μl each) by spotting 1, 2, 3 and 20′ drops and allowed to air dry in a laminar flow hood. Cas9-RNPs were precipitated onto 0.6 μm gold particles (Bio-Rad, Hercules, CA) using the cationic lipid polymer, TransIT-2020 (Mirus, Madison, WI). After loading 10 μl of gold particle suspension to each microcarrier, time needed to dry particle suspension was measured. In addition, another 10 μl of gold particle suspension onto microcarrier disk after drying the first spreading of 10 μl of gold particle suspension is loaded.

### DsRED Protein or RNP Bombardment

Four hours prior to bombardment, IEs were placed on 55 mm filter paper in the center of DBC3 osmoticum media supplemented with 0.2 M mannitol and 0.2 M sorbitol. Using prepared microcarriers holding DsRED- or RNP-coated gold microparticles, IEs for each sample were shot once using the PDS-100/He gene gun (Bio-Rad, Hercules, CA) with rupture pressure of 1100 psi (Table 1) and transferred from the filter paper directly to the media below and incubated at 28° C. for 20 hours. Bombarded IEs were then transferred to DBC3 media without osmoticum and incubated in the dark 28° C.

TABLE 1
Experimental Design for Effect of Number of Drops of
Protein-coated Gold Microparticle Suspension per Volume
on Drying Time and Macrocarrier-covering Area.

# of DsRED or RNP drops

| | 1 | 20 or more |
|---|---|---|
| Time needed to dry particle | >120 min | 15-20 min |
| suspension (min) | | |
| Particle covering area | + | ++(+) |

### Fluorescent Visualization

Fluorescent images of IEs were visualized to evaluate tissue damage, # of DsRED foci and distribution/covering area of DsRED foci on individual IEs using a fluorescent Leica M165 FC stereomicroscope, equipped with Leica DFC7000 T (JH Technologies, Fremont, CA), using two microscopic filters, brightfield and ET DSR with 545 nm excitation and 620 nm emission. The microscope is linked to a camera imaging software, Leica Application Suite version 4.9, which was used to capture the fluorescent images. Tissue damage and DsRED fluorescence were measured at different magnifications at 1 to 3 days after bombardment.

### Results

It took only 15-20 min to air dry 10 μl of RNP-coated gold particle suspension completely when 10 μl of particle suspension was spotted with 20 or more drops onto a microcarrier while it took 2 hours or longer with 1 drop spotting (Table 1). Others reported a similar result where they allowed to air-dry at 22° C. for 16 hours when 12 μl of DsRed protein solution was spread directly onto the macrocarrier set. However, they did not test multiple drops. In addition, our study showed that spotting multiple small drops of particle suspension was less sticky, covered more area of target immature embryos more uniformly, compared to spotting a single large drop (Table 1).

### Example 3

### Effect of Number of Protein-coated Gold Microparticle Bombardments on Protein Delivery Efficiency and Target Tissue Damage

### Plant Materials and Production of Embryogenic Callus Tissues

Seeds of Triticum aestivum L. cv. Fielder were sown weekly and grown in growth chambers under 16-hr days at 24° C., and 8-hr nights at 15° C. Light levels were set to approximately 130 μmol m⁻² s⁻¹ at head height. Immature spikes were harvested 10-14 days post flowering with an immature embryo (IE) sized 1.7-2.2 mm. Immature spikes were collected up to 5 days pre bombardment and stored at 4-6° C. One day prior to bombardment, immature seeds were harvested from immature spikes and surface-sterilized using 20% (v/v) bleach (8.25% sodium hypochlorite) plus one drop of Tween 20 for 15 minutes before triple rinsing with sterile water. IEs were then isolated and placed scutellum side up on DBC3 medium and incubated at 26° C. overnight.

### Gold Particle Preparation for Bombardment

Purified DsRED protein were precipitated onto 0.6 μm gold particles (Bio-Rad, Hercules, CA) with or without the cationic lipid polymer, TransIT-2020 (Mirus, Madison, WI). Briefly, for 3 shots, 60 μl of purified DsRED protein at 0.1 μg/μl was mixed gently with 30 μl sterile gold particles (10 μg μl⁻¹ water suspension)+/−1.5 μl TransIT-2020. The DsRED gold particles were pelleted in a mini microcentrifuge at 2,000 g for 30 s. The supernatant was removed, and the gold particles were resuspended in 30 μl of sterile water by brief sonication. The coated gold particles were immediately applied to 3 macrocarriers (10 μl each) by spotting numerous small drops and allowed to air dry in a laminar flow hood.

### DsRED Protein Bombardment

Four hours prior to bombardment, IEs were placed on 55 mm filter paper in the center of DBC3 osmoticum media supplemented with 0.2 M mannitol and 0.2 M sorbitol. Using prepared microcarriers holding DsRED-coated gold microparticles, IEs for each sample were shot once to five times using the PDS-100/He gene gun (Bio-Rad, Hercules, CA) with rupture pressure of 1100 psi and transferred from the filter paper directly to the media below and incubated at 28° C. for 20 hours. Bombarded IEs were then transferred to DBC3 media without osmoticum and incubated in the dark 28° C.

### Fluorescent Visualization

Fluorescent images of IEs and calli were visualized with a fluorescent Leica M165 FC stereomicroscope, equipped with Leica DFC7000 T (JH Technologies, Fremont, CA), using two microscopic filters, brightfield and ET DSR with 545 nm excitation and 620 nm emission. The microscope is linked to a camera imaging software, Leica Application Suite version 4.9, which was used to capture the fluorescent images. Tissue damage and screening of fluorescent activity were measured at different magnifications at 1 to 7 days after bombardment.

### Example 4

### RNP Delivery to Rice Callus Tissues and Generation of Edited Plants

### Plant Materials and Production of Embryogenic Callus Tissues

Rice cultivar Nipponbare (Oryza sativa ssp. Japonica) plants were grown in a soil mixture comprised of equal parts turface and sunshine mix #4 in the greenhouse with temperature setpoints of 27° C./22° C. at ambient light conditions with daylengths of 12 hr in Kord 6-inch, 1.835 L pots. All plants were fertilized with 125 mL of 1% w/v iron solution one-week post-transplant. 1000 mL of 5% w/v JR Peter's Blue 20-20-20 fertilizer was added to each flat at 3- and 11-weeks post-germination. Well-watered plants were provided a constant supply of water by maintaining a flooded condition in the tray. Immature panicles were harvested 12-15 days post flowering with an immature embryo (IE) sized 1.8-2.5 mm. Immature rough kernels were collected from immature panicles and surface-sterilized using 70% ethanol for 5 min and rinsed with sterile water. The kernels were then 20% (v/v) bleach (8.25% sodium hypochlorite) plus one drop of Tween 20 for 15 minutes before triple rinsing with sterile water. IEs were then isolated and placed scutellum side up on OsCIM2 callus induction medium [N6 salts and vitamins, 30 g/L maltose, 0.1 g/L myo-inositol, 0.3 g/L casein enzymatic hydrolysate, 0.5 g/L L-proline, 0.5 g/L L-glutamine, 2.5 mg/L 2,4-D, 0.2 mg/L BAP, 5 mM CuSO₄, 3.5 g/L Phytagel, pH 5.8] and incubated in the dark at 28° C. to initiate callus induction. Six- to 8-day-old embryogenic calli were used as targets for RNP bombardment.

### Cas9-gRNA RNP Assembly

A 1:1 ratio of tracrRNA and target specific crRNA (Integrated DNA Technologies, Coralville, IA) were annealed to form afunctional gRNA duplex. Cas9 protein with a C-terminal double nuclear-localization tag (QB3 Macrolab, Berkeley) and sgRNAs were complexed in vitro to form Cas9-gRNA RNPs. For each sample bombardment with 2 shots, a 40 ul reaction was assembled. Thoroughly mixed were 3.0 μg sgRNA, 4 μl 10×NEBuffer 3.1, and nuclease-free water. Then, in a drop-wise manner, 3.2 μg Cas9 was added slowly with constant mixing, followed by 20 min incubation at 37° C. The resultant RNP mixtures were stored on ice until gold particle prep.

### Gold Particle Preparation for Bombardment

Cas9-RNPs were precipitated onto 0.6 μm gold particles (Bio-Rad, Hercules, CA) using the cationic lipid polymer, TransIT-2020 (Mirus, Madison, WI). Briefly, for 2 shots, 40 μl Cas9-RNP mixture, as described above, was mixed gently with 20 μl sterile gold particles (10 μg 1-water suspension) and 1.0 μl TransIT-2020 and incubated on ice for 20 min. The Cas9-RNP coated gold particles were pelleted in a mini microcentrifuge at 2,000 g for 30 s. The supernatant was removed, and the gold particles were resuspended in 20 μl of sterile water by brief sonication. The coated gold particles were immediately applied to 2 macrocarriers (10 μl each) by spotting numerous small drops and allowed to air dry in a laminar flow hood. Each plate was bombarded twice using the 2 prepared macrocarriers.

### RNP Bombardment and Regeneration

Four hours prior to bombardment, calli were placed on 55 mm filter paper in the center of OsCIM2 osmoticum media supplemented with 0.2 M mannitol and 0.2 M sorbitol. Using two prepared microcarriers holding Cas9-RNP coated gold microparticles, tissues for each sample were shot twice using the PDS-100/He gene gun (Bio-Rad, Hercules, CA) with rupture pressure of 1100 psi. The bombarded tissues were transferred from the filter paper directly to the media below and incubated at 28° C. for 20 hours. Bombarded FEC issues were transferred to OsCIM2 media in the dark 28° C. for 8 weeks with subculturing as needed. The tissues were then transferred to OsReg regeneration medium [MS salts and vitamins, 30 g/L sucrose, 30 g/L sorbitol, 0.5 mg/L NAA and 1 mg/L BAP] and incubated at 28° C., 16-hr light, 90 mmol m⁻² s⁻¹. When regenerated plantlets reached at least 1 cm in height, they were transferred to rooting medium (MS salts and vitamins, 20 g/L sucrose, 1 g/L myo-inositol) and incubated at 28° C. under conditions of 16-hr light (150 mmol m⁻² s⁻¹) and 8-h dark until roots were established and leaves touched the Phytatray™ lid. Plantlets were then transferred to soil.

### Detection of CRISPR/Cas9 Mutations

Genomic DNA was extracted from leaf tissue of individual plants from the pools (groups) with positive Amplicon sequencing results following a CTAB extraction method. PCR amplifications using a primer set, Pi21 F4 (5′-GACCAAAGCCTGTCTATCTGCATT-3′ SEQ ID NO:9) and Pi21 R6 (5′-AGACGTCGACGATGAGGATCT CCT-3′ SEQ ID NO:10) to amplify 471-bp fragments were performed in a 25-μl reaction with DreamTaq PCR Master Mix (2×) (Thermo Fisher Scientific, Grand Island, NY). For each PCR reaction, 23 μL were loaded onto a 0.8% agarose gel for electrophoresis. The amplified PCR product was cut, and DNA was extracted from gel samples using the Qiagen QIAquick® Gel Extraction Kit (QIAGEN, Chatsworth, CA). The purified DNA samples were used for Sanger sequencing to determine mutation patterns.

### Results

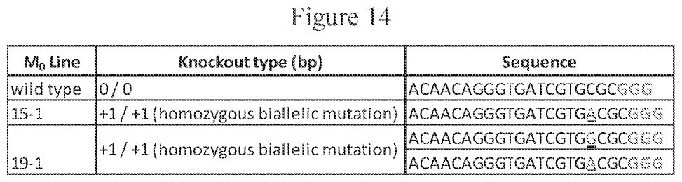

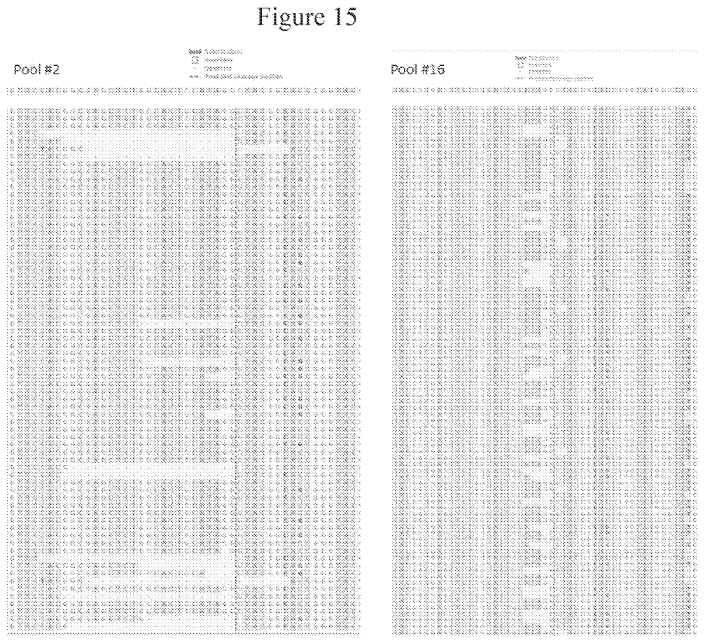

In total over 100 M₀ rice plants were generated from RNP-bombarded rice calli induced from immature embryos. Genomic DNA was extracted from leaf samples randomly selected from 29 M₀ rice plants and used for Sanger sequencing to detect mutation patterns. Two out of 29 plants showed mutations, giving a 6.9% mutation efficiency. One plant had a homozygous biallelic mutation (1-bp insertion) while another had a heterozygous biallelic mutation (G insertion and A insertion on each locus) (FIG. 14).

### Example 5

### RNP Delivery to Wheat Immature Embryos and Generation of Edited Plants

### Plant Materials and Production of Embryogenic Callus Tissues

Seeds of Triticum aestivum L. cv. Fielder were sown weekly and grown in growth chambers under 16-hr days at 24° C., and 8-hr nights at 15° C. Light levels were set to approximately 130 μmol m⁻² s⁻¹ at head height. Immature spikes were harvested 10-14 days post flowering with an immature embryo (IE) sized 1.7-2.2 mm. Immature spikes were collected up to 5 days pre bombardment and stored at 4-6° C. One day prior to bombardment, immature seeds were harvested from immature spikes and surface-sterilized using 20% (v/v) bleach (8.25% sodium hypochlorite) plus one drop of Tween 20 for 15 minutes before triple rinsing with sterile water. IEs were then isolated and placed scutellum side up on DBC3 medium, incubated at 26° C. overnight, and used as targets for RNP bombardment.

### Cas9-gRNA RNP Assembly

A 1:1 ratio of tracrRNA and target specific crRNA (Integrated DNA Technologies, Coralville, IA) were annealed to form a functional gRNA duplex. Cas9 protein with a C-terminal double nuclear-localization tag (QB3 Macrolab, Berkeley) and sgRNAs were complexed in vitro to form Cas9-gRNA RNPs. For each sample bombardment with 2 shots, a 40 μl reaction was assembled. Thoroughly mixed were 3.0 μg sgRNA, 4 μl 10×NEBuffer 3.1, and nuclease-free water. Then, in a drop-wise manner, 3.2 μg Cas9 was added slowly with constant mixing, followed by 20 min incubation at 37° C. The resultant RNP mixtures were stored on ice.

Gold Particle Preparation for Bombardment Cas9-RNPs were precipitated onto 0.6 μm gold particles (Bio-Rad, Hercules, CA) using the cationic lipid polymer, TransIT-2020 (Mirus, Madison, WI). Briefly, for 2 shots, 40 μl Cas9-RNP mixture, as described above, was mixed gently with 20 μl sterile gold particles (10 μg μl⁻¹ water suspension) and 1.0 μl TransIT-2020 and incubated on ice for 20 min. The Cas9-RNP coated gold particles were pelleted in a mini microcentrifuge at 2,000 g for 30 s. The supernatant was removed, and the gold particles were resuspended in 20 μl of sterile water by brief sonication. The coated gold particles were immediately applied to 2 macrocarriers (10 μl each) by spotting numerous small drops and allowed to air dry in a laminar flow hood. Each plate was bombarded twice using the 2 prepared macrocarriers.

### RNP Bombardment and Regeneration

Four hours prior to bombardment, IEs were placed on 55 mm filter paper in the center of DBC3 osmoticum media supplemented with 0.2 M mannitol and 0.2 M sorbitol. Using two prepared microcarriers holding Cas9-RNP coated gold microparticles, IEs for each sample were shot twice using the PDS-100/He gene gun (Bio-Rad, Hercules, CA) with rupture pressure of 1100 psi. The bombarded tissues were transferred from the filter paper directly to the media below and incubated at 28° C. for 20 hours. Bombarded IEs were transferred to DBC3 media without osmoticum in dim light (10-30 μmol m⁻² s⁻¹) at 28° C. for 9 weeks with subculturing as needed. Highly regenerative tissues originating from each IE was transferred to DBC6 media for regeneration. Resultant plantlets were transferred to rooting media and incubated in high light (90-100 μmol m⁻² s⁻¹) at 28° C. and grown to 10-15 cm before being transplanted to soil.

### Amplicon Next Generation Sequencing Analysis

To determine mutation rates for the pooled regenerated plantlets 3-4 months after bombardment by amplicon sequencing, PCR was performed with target-specific primers, (forward primer: 5′-GCTCTTCCGATCTccatgtcacccgaagccatg-3′ SEQ ID NO:11 and reverse primer: 5′-GCTCTTCCGATCTgcgactgcgagcacttacag-3′ SEQ ID NO:12) amplifying approximately 370 bp around the cut site using Phusion High Fidelity (New England Biolabs, Ipswich, MA) polymerase. Primers contained a 5′-stub compatible with Illumina NGS library preparation. PCR products were ligated to Illumina TruSeq adaptors and purified. Libraries were prepared using a NEBNext kit (Illumina, San Diego, CA) according to the manufacturer's guidelines. Samples were deep sequenced on an Illumina iSeq at 200-300 bp paired end reads to a depth of approximately 10,000 reads per sample. Cortado was used to analyze editing outcomes. Briefly, reads were adapter trimmed then merged using overlap to single reads. These joined reads were then aligned to the target reference sequence.

### Detection of CRISPR/Cas9 Mutations

Genomic DNA was extracted from leaf tissue of individual wheat plants from the pools (groups) with positive Amplicon sequencing results following a CTAB extraction method. PCR amplifications using homoallele specific primers across each genome (Table 2) were performed in a 25-μl reaction with DreamTaq PCR Master Mix (2×) (Thermo Fisher Scientific, Grand Island, NY). For each PCR reaction, 23 L were loaded onto a 0.8% agarose gel for electrophoresis. The amplified PCR product was cut, and DNA was extracted from gel samples using the Qiagen QIAquick Gel Extraction Kit (QIAGEN, Chatsworth, CA). The purified DNA samples were used for Sanger sequencing to determine mutation patterns.

TABLE 2
Primer sets used for screening of genome-edited plants derived from RNP
bombardment

| | | | Band |
|---|---|---|---|
| | | | Size |
| Genome | Name | Sequence | (bp) |
| A genome | Pi21(A)11F | AACTATGATTGCTTTGCCGG (SEQ ID NO: 13) | 985 |
| | Pi21(A)10R | GGCGGCCAGCAATTAGCA (SEQ ID NO: 14) | |
| B genome | Pi21(B)4F | TGAAAATACCATTGCTTGGTCA (SEQ ID NO: 15) | 1,211 |
| | Pi21(B)113R | CACGTCAATGCAGTCCAAAGC (SEQ ID NO: 16) | |
| D genome | Pi21(D)8F | CTCGGTGCGATATGACCGAATAA (SEQ ID NO: 17) | 1,245 |
| | Pi21(D)12R | CCACATCAATGTAGTCCATGC (SEQ ID NO: 18) | |

### Results

Amplicon sequencing results showed that 14 out of 31 M₀ plant pools had mutations (Table 3); each pool contained 5 to 15 plants.

TABLE 3
Amplicon sequencing results from 31 M₀ plant pools

| | Mutation |
|---|---|
| Pool # | efficiency |
| 2 | ++++ |
| 3 | + |
| 4 | ++++ |
| 5 | +++ |
| 6 | ++ |
| 7 | − |
| 9 | − |
| 13 | +++++ |
| 14 | ++++ |
| 15 | + |
| 16 | +++++ |
| 17 | ++++ |
| 18 | ++ |
| 19 | ++++ |
| 20 | +++ |
| 21 | − |
| 23 | − |
| 24 | − |
| 25 | − |
| 26 | − |
| 30 | − |
| 31 | − |
| 32 | +++ |
| 35 | − |
| 36 | − |
| 37 | − |
| 38 | − |
| 39 | − |
| 40 | − |
| 41 | − |
| 42 | − |

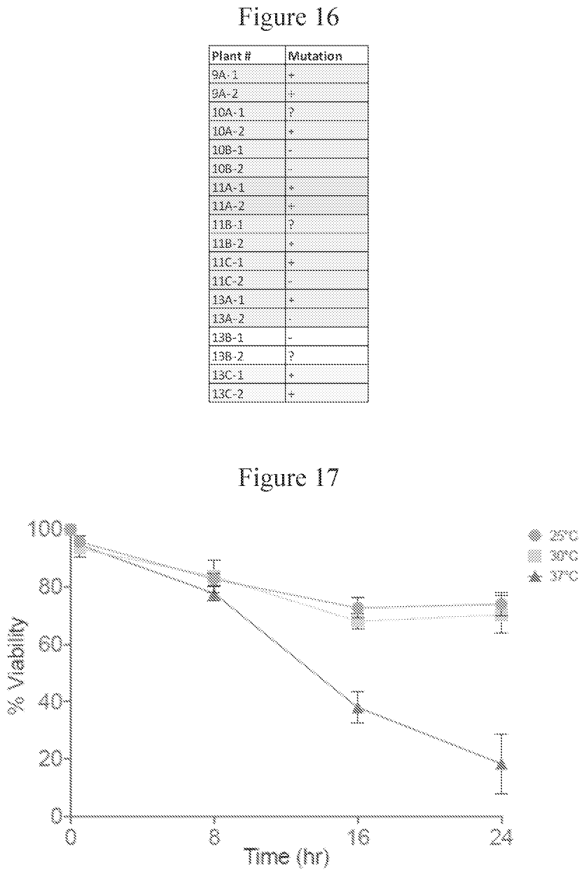

Eighteen plants collected from the 3 highest mutation pools were independently genotyped by Sanger sequencing; at least 10 plants had a mutation (FIG. 16).

### Example 6

### Impact of Temperature and Time on DNA-Free Cas9-Ribonucleoprotein Mediated Gene Editing in Wheat Protoplasts and Immature Embryos

### Summary

The advancement of precision engineering for crop trait improvement is important in the face of rapid population growth, climate change, and disease. To this end, targeted double-stranded break technology using RNA-guided Cas9 has been adopted widely for genome editing in plants. Agrobacterium or particle bombardment-based delivery of plasmids encoding Cas9 and guide RNA (gRNA) is common, but requires optimization of expression and often results in random integration of plasmid DNA into the plant genome. Recent advances have described gene editing by the delivery of Cas9 and gRNA as pre-assembled ribonucleoproteins (RNPs) into various plant tissues, but with moderate efficiency in resulting regenerated plants. In this report we describe improvements to Cas9-RNP mediated gene editing in wheat. We demonstrate that Cas9-RNP assays in protoplasts are a fast and effective tool for rational selection of optimal gRNAs for gene editing in regenerable immature embryos (IEs), and that high temperature treatment enhances gene editing rates in both tissue types. We also show that Cas9-mediated editing persists for at least 14 days in gold particle bombarded wheat IEs. The regenerated edited wheat plants in this work are recovered at high rates in the absence of exogenous DNA and selection. With this method, we produce knockouts of a set of three homoeologous genes as well as two pathogenic effector susceptibility genes that result in insensitivity to corresponding necrotrophic effectors produced by Parastagonospora nodorum. The establishment of highly efficient, DNA-free gene editing technology holds promise for accelerated trait diversity production in an expansive array of crops.

### Introduction

Amidst a rapidly growing population and threats posed by climate change and disease, there exists a need for the advancement of crop biotechnology to increase the speed and precision of crop varietal development. Cas9 has emerged as a plant gene editing tool of choice for its accuracy and programmability to engineer allelic diversity for beneficial traits to support global food security. Guided by RNA, Cas9 efficiently makes sequence-specific double-stranded breaks in genomic DNA. The host's double-stranded break repair mechanisms are then elicited. Non-homologous end joining (NHEJ), the predominant and often error prone pathway in plants, can lead to insertions or deletions (indels) at the Cas9 cut site upon repair. Exploitation of this system allows for targeted knockout of endogenous genes.

Cas9 and guide RNA (gRNA) encoding plasmid DNA systems have been developed and delivered to plant and major crop species including Arabidopsis, potato, tomato, soybean, maize, barley, rice, and wheat by Agrobacterium tumefaciens or particle bombardment. These methods rely on random integration of Cas9-gRNA cassettes into the genome, and optimization of expression for each plant system. As a result, the gene editing process is encumbered by variables such as promoter and terminator choice when cloning constructs and copy number and integration location of transgenes upon transformation. Additionally, gene editing by these methods raises transgenic regulatory concerns. Regulation aside, transgenes can often be segregated away through breeding, but the process is laborious, time consuming, and particularly difficult for plants with complex genomes. Moreover, crops with lengthy generation times or those that are vegetatively propagated, such as cassava and banana, cannot be bred to segregate transgenes. There have been reports in which plant gene editing has been achieved by transient expression of Cas9 and gRNA, however full experimental control over the fate of transgene integration and tracking has not been achieved. For these reasons, the advancement of DNA-free genome editing technology can be useful.

The direct delivery of preassembled Cas9-gRNA ribonucleoproteins (RNPs) is one such technology and has been demonstrated in various plant protoplast systems to induce targeted mutations. Some have produced edited plants arisen from the transfected single cells, however regeneration of wheat and other crop plant protoplasts is not feasible with current methods. Cas9-RNP based editing of maize, rice, and wheat regenerable embryos by biolistics has also been reported. Gold particles coated with Cas9-RNPs are bombarded with high pressure into immature embryos (IEs) that are ultimately regenerated into plants through tissue culture. Co-delivery of DNA vectors with selective markers or helper genes along with Cas9-RNPs have been utilized to improve editing efficiency. In the absence of selection, however, editing rates have generally been low.

The use of Cas9-RNPs to generate edited plants provides unique benefits. Because the gene editing reagents are delivered as pre-assembled complexes, researchers do not need to optimize DNA vectors, the host plant tissue does not bear the burden of transcribing or translating Cas9 or gRNA, and breeding for segregation is unnecessary due to the absence of transgenes. Additionally, the Cas9-RNPs, which exist in a finite amount in the target tissue, are ultimately degraded by endogenous proteases and nucleases. However, there remains room to improve the editing pipeline and increase efficiency.

Low rates of Cas9 mediated editing in plant tissue may indicate that the endonuclease is not reaching its full potential due to suboptimal environmental conditions. Studies across organisms including Arabidopsis, citrus, and wheat have shown that Cas9 generates more targeted indels at elevated temperatures.

Here, we present advances in Cas9-RNP based gene editing in the global food crop, wheat. To determine if temperature can be harnessed to enhance Cas9-RNP mediated editing, we explore the effects of heat treatment on transfected wheat protoplasts and IEs. We examine the relationship of editing efficiency between non-regenerable protoplasts and regenerable IEs and monitor the rate of editing over time. We demonstrate that treatment at elevated temperatures increases gene editing efficiency in both tissue systems and find that the RNP transfection technique of gold particle bombardment results in sustained editing of tissue at least 14 days after bombardment. We also find that editing rates in protoplasts correlate linearly with editing rates in IEs. Therefore, rapid in vivo protoplast assays can be instituted as a standard gene editing pipeline step to select the most effective gRNAs for IE gene editing and regeneration. Lastly, we regenerate wheat plants edited via Cas9-RNP biolistic transfection. As a proof of method, we simultaneously target three wheat homoeologous orthologs of a rice gene, Pi21(Os04 g0401000), and successfully generate lines with knockouts in all copies. We also target wheat genes Tsn1 and Snn5, producing lines that are insensitive to the Parastagonospora nodorum pathogenic effectors SnToxA and SnTox5 and establish DNA and selection-free Cas9-RNP mediated editing as an efficient and feasible technique for generating targeted gene knockouts in wheat.

### Results

### Cas9-RNP Transfection and the Effect of Temperature in Wheat Protoplast Gene Editing

We first quantified cell viability after heat treatment of non-transfected protoplasts to determine the feasibility of testing higher temperatures for wheat protoplast gene editing. Protoplasts were isolated from partially etiolated wheat seedlings and incubated at 25° C., 30° C., or 37° C. for 16 hours followed by 25° C. for 8 hours. During the 24-hour period, the protoplasts were monitored for viability every 8 hours using Evans blue staining and microscopy. Viability of protoplasts treated at 37° C. decreased markedly compared to those treated at 25° C. and 30° C. (FIG. 17) and suffered from media evaporation. It was therefore concluded that the protoplast gene editing pipeline is not amenable to a 37° C. heat treatment.

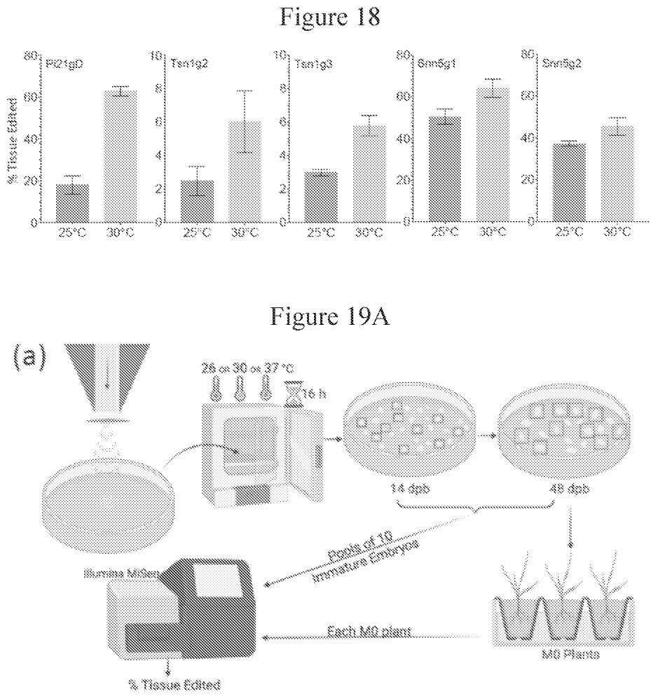

Five single guide RNAs (sgRNAs), Pi21gD, Tsn1 g2, Tsn1 g3, Snn5 g1, and Snn5 g2 were selected and commercially synthesized for this study. To assess the efficacy of the sgRNAs in vivo, and to determine the effect of temperature on wheat protoplast gene editing, Cas9-RNPs were assembled and transfected into wheat mesophyll protoplasts. Purified Cas9 with a C-terminal double nuclear-localization tag was complexed with sgRNA. The resulting sgRNA-Cas9 RNPs were transfected into wheat protoplasts using polyethylene glycol (PEG). Transfected protoplasts were treated at 25° C. or 30° C. and harvested for genotypic analysis after 24 hours. Editing rates at the target loci were determined by amplicon next-generation sequencing (NGS). With incubation at 25° C. and 30° C., average editing rates ranged from 2.5-50% and 5.8-62% respectively. Despite this variability between different sgRNA-Cas9 RNPs, editing efficiency was consistently higher in protoplasts treated at 30° C. compared to 25° C. for any given sgRNA (FIG. 18), suggesting that a higher temperature treatment is advantageous to RNP-mediated gene editing in wheat protoplasts.

### Biolistic Cas9-RNP Delivery and the Effect of Temperature in Wheat Immature Embryo Gene Editing

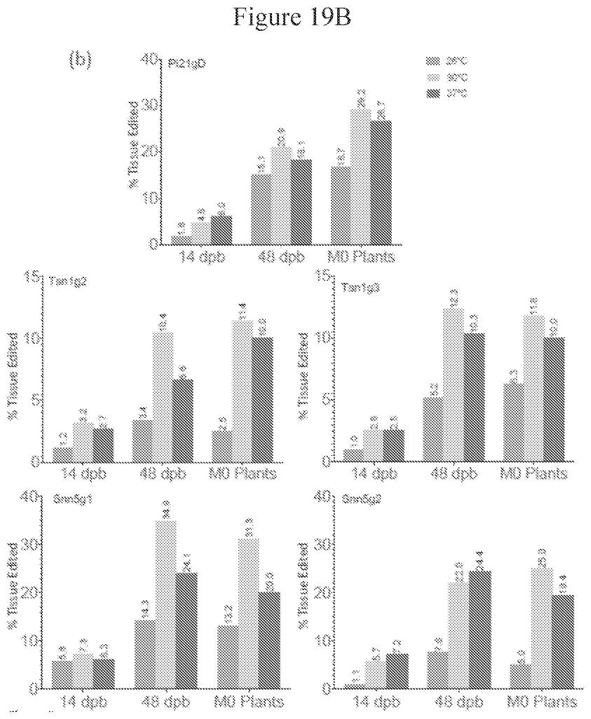

To determine if a high temperature treatment similarly improves Cas9-RNP based editing in wheat IEs as it does in protoplasts, RNPs were transfected into IEs by particle bombardment. The experimental pipeline is summarized in FIG. 19A. Single guide RNA and Cas9 were complexed in vitro, adsorbed onto 0.6 μm gold particles, and biolistically delivered with a helium-pressured particle gun. For each sgRNA and temperature being tested, 30 IEs were bombarded and incubated at 26° C., 30° C., or 37° C. for 16 hours. They were then maintained at 26° C. on callus-induction media before inducing regeneration at around 63 days post-bombardment (dpb). Plasmid DNA was not co-delivered with any of the Cas9-RNPs, and callus induction and regeneration were performed under selection-free conditions. From each set of 30 RNP-transfected embryos, ten were randomly harvested and pooled for genomic analysis at 14 dpb and again at 48 dpb. The remaining ten embryos were kept for regeneration into M₀ plants. All independent shoots were isolated and treated as individual M₀ plants. Plants were transplanted from tissue culture media to soil approximately 100 dpb. Each resulting M₀ plant was independently genotyped, and the percent tissue edited rate was calculated as the percentage of mutant alleles among total alleles in the M₀ plant pool. The percentage of plants edited was also calculated as a percentage of the number of plants with any edit among the number of total M₀ plants regenerated. All genomic analysis was done by amplicon NGS.

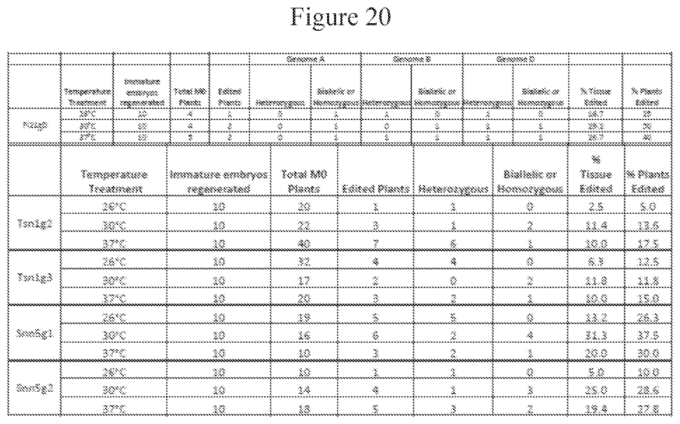

Elevated temperature treatment of both 30° C. and 37° C. led to higher percentages of edited tissue compared to 28° C. for all five sgRNA-Cas9 RNPs across all timepoints (FIG. 19B). Tissue editing rates were higher at 48 dpb than at 14 dpb and editing rates in the M₀ regenerant tissue pool were comparable to those at 48 dpb. From the ten embryos per treatment allowed to regenerate, 10-40 M₀ plants were produced. Plants with wild type, heterozygous, biallelic, and homozygous mutations at the target loci were obtained. Editing efficiency in the M₀ regenerants is summarized in FIG. 20.

### Cas9-RNP Mediated Editing is Sustained Over Time

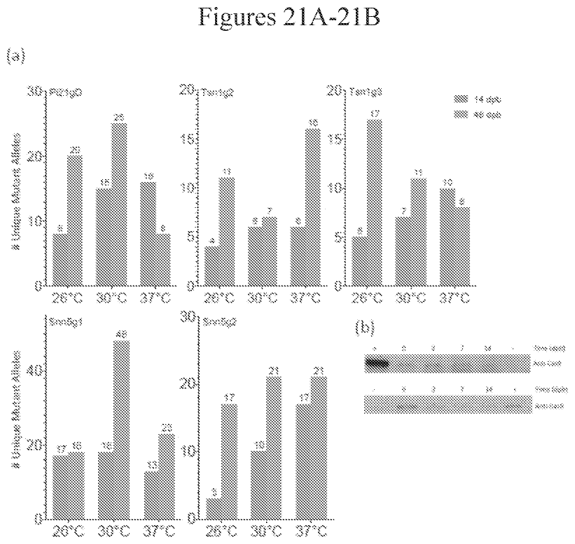

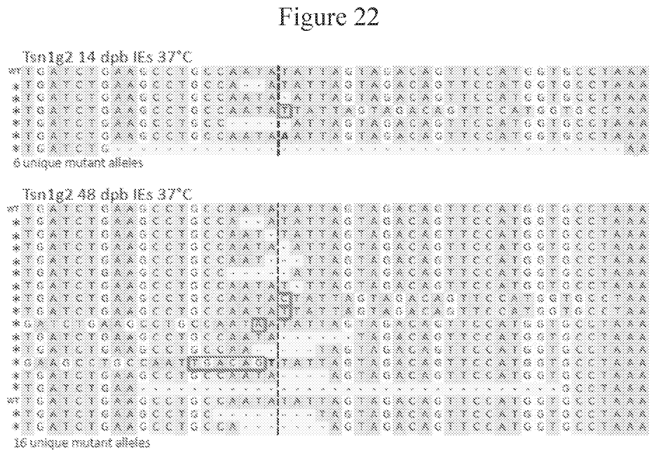

Notably, gene editing rates were more than doubled, regardless of temperature treatment, in tissue assayed at 48 dpb compared to 14 dpb (FIG. 19B). To further investigate the difference in editing rates over time, the number of unique mutant alleles was determined at the 14 and 48 dpb timepoints. With minimal exception, there were more unique mutant alleles at 48 dpb compared to 14 dpb (FIG. 21A, FIG. 22).

An additional 50 IEs were bombarded with Snn5 g1-Cas9 RNP to determine the length of time that Cas9 remains present in biolistically transfected tissue. Western blot analysis was performed with 10-embryo tissue samples taken 0, 2, 7, and 14 dpb. Given the finite amount of Cas9 protein delivered by RNP bombardment and rapid cell division and growth in each IE over time, we normalized the experiment by volume extracted from total tissue originating from ten IEs at any given timepoint, rather than total protein extracted. Cas9 was detected in tissue from all four timepoints with decreasing band intensity over time (FIG. 21B). Cas9 was not detected in embryos that were not subjected to bombardment of Cas9-RNPs. Due to the large mass of tissue from exponential growth of callus from IEs, it was not feasible to extract protein from and perform Western blot analysis on ten-embryo 48 dpb samples. Taken together, these results suggest that Cas9 mediated editing activity is sustained over the course of at least 14 days after biolistic delivery of Cas9-RNPs into immature wheat embryos. When using this method, the degradation of Cas9 protein in the target tissue is not as rapid as previously hypothesized, and evaluation of editing efficiency should occur 14 to 48 dpb for increased accuracy.

Relative Editing Rates in Protoplasts Correlate Linearly with Editing Rates in M₀ Regenerants from Bombarded Immature Embryos

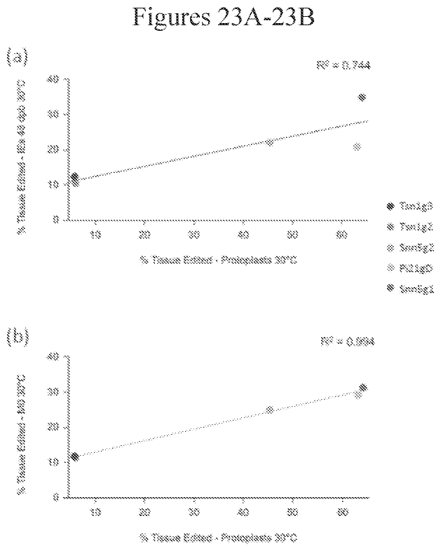

The different sgRNA-Cas9 RNPs used in this study conferred different levels of efficacy in both PEG transfected protoplasts and biolistically transfected embryos. To determine whether the editing rates in the two tissue systems correlated with one another, each sgRNA-Cas9 RNP's average editing efficiency in 30° C. treated protoplasts was plotted against its editing efficiency in 48 dpb 30° C. treated bombarded IEs as well as the M₀ 30° C. treated regenerant tissue pool. A linear regression model was applied to the data, revealing a positive linear correlation with R²=0.744 and R²=0.994, respectively (FIG. 23A-23B). Though a survey of a greater number of sgRNAs would strengthen this association, the present data suggest that editing efficiency in protoplasts can be predictive of editing efficiency in IEs. Given the positive correlation between RNP-mediated editing rates in protoplasts and in biolistically transfected IEs, it can be beneficial to first rapidly score the efficiency of various gRNA candidates in protoplasts to optimize for the highest rate of edited regenerant tissue.

Cas9-RNP Mediated Knockout of Parastagonospora nodorum Necrotrophic Effector Sensitivity Genes

In this study, 20 M₀ Tsn1 edited plants were produced from 30 transfected embryos maintained for regeneration. Of those, 14 had heterozygous mutations and 6 had biallelic or homozygous mutations. Fully expanded secondary leaves of a subset of M₀ Tsn1 edited plants, M₀ Tsn1 WT plants, and Fielder grown from seed were infiltrated with SnToxA expressed in Pichia pastoris. After 72 hours, M₀ heterozygotes, M₀ WT, and Fielder plants had necrotic lesions extending from the site of infiltration. Meanwhile, M₀ plants with biallelic or homozygous mutations exhibited no necrosis (FIG. 24A-24M).

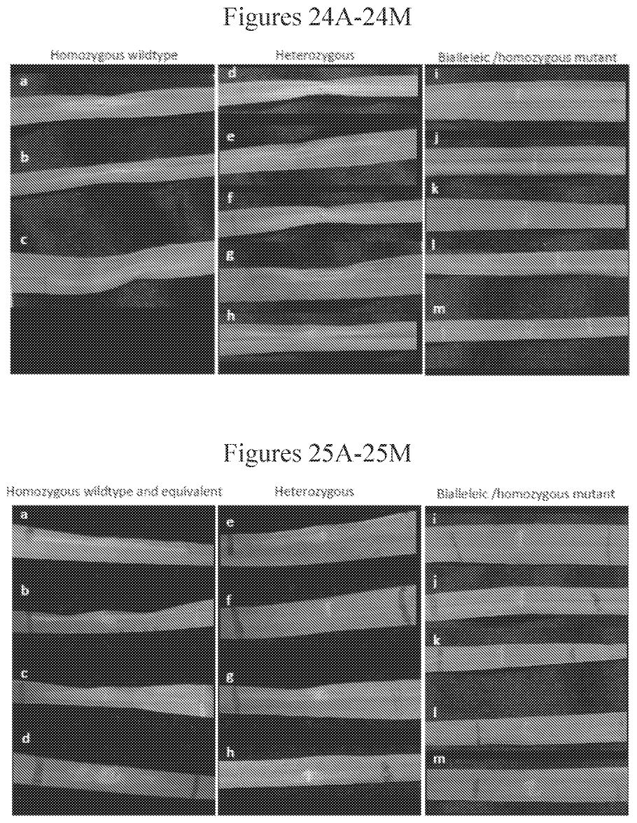

Similarly, a total 24 M₀ Snn5 edited plants were produced from 30 transfected embryos maintained for regeneration. Of those, 14 had heterozygous mutations and ten had biallelic or homozygous mutations. Fully expanded secondary leaves of a subset of M₀ Snn5 edited plants, M₀ Snn5 WT plants, and Fielder grown from seed were infiltrated with SnTox5 containing culture filtrates. After 72 hours, M₀ heterozygotes with in-frame deletions, M₀ WT, and Fielder plants exhibited necrotic lesions. Results for M₀ heterozygotes, however, displayed a mixture of phenotypes ranging from sensitive to insensitive. Two heterozygous plants with an in-frame deletion on one allele appeared insensitive to SnTox5. Notably, all plants with biallelic or homozygous mutations leading to premature termination were insensitive to SnTox5 (FIG. 25A-25M).

These results demonstrate that loss-of-function mutations can be introduced to both copies of a gene within the M₀ generation, leading to insensitivity to agronomically relevant necrotrophic fungal effectors. M₀ heterozygotes and biallelic plants can be self-fertilized to establish lines with homozygous deleterious mutations in the susceptibility genes. The biolistic method with 30° C. or 37° C. heat treatment is highly efficient, and edited plants can be identified from a small number of regenerants without the use of selection in tissue culture.

### Discussion

CRISPR-based RNPs have been used for editing in various plant species and tissue types. In this work, we improve upon DNA-free Cas9-RNP technology for genome editing in wheat. We establish heat treatment as a parameter to increase the rate of editing in vivo, show that particle bombardment-based editing is sustained over more than 14 days, and demonstrate that results from protoplast assays can be utilized as a proxy for predicting editing rates in regenerable tissue and as a tool to rank gRNA efficacy. By delivering gene editing reagents as protein-RNA complexes, several complications associated with Agrobacterium tumefaciens and biolistic DNA vector delivery are avoided.

Cas9 from Streptococcus pyogenes, a bacterium that grows optimally at 37° C., has been shown to exhibit increased cleavage activity at 370 compared to 22° C. in vitro. Plant protoplast and IE transfections and regeneration are typically performed at ambient temperatures (25° C. and 26° C. respectively). Although modulation of temperature has not been previously performed in protoplast gene editing experiments, an increase in temperature for DNA-based plant gene editing studies have resulted in higher targeted mutation frequencies. The application of temperature treatment to increase Cas9-RNP mediated editing efficiency in any plant tissue system has not previously been demonstrated. Here, we found that 16 hours of exposure of Cas9-RNP transfected protoplasts to 30° C. markedly increased indel formation at the Cas9 cut site (FIG. 18). Similarly, 16 hours of exposure of Cas9-RNP bombarded IEs to 30° C. or 37° C. resulted in increased targeted indel formation. In IEs assayed at 48 dpb we achieved editing rates of 10.4-34.9% with 30° C. treatment, 6.63-24.39% with 37° C. treatment, and just 3.36-14.25% with standard 26° C. incubation (FIG. 19B). Interestingly, the benefit of increased temperature treatment was consistent between the two target tissues and across the 5 different target sites tested. In our work, there were no discernable defects in regenerability for IEs treated at a higher temperature compared to the standard 26° C. We detected no positive or negative correlation between temperature treatment and the number of M₀ plants recovered.

Previous reports have described the biolistic delivery of Cas9-RNPs into wheat and maize embryo cells in the absence of DNA and selection. Both achieved moderate targeted mutagenesis frequencies in the regenerated plants. We noted that the studies each assayed for editing efficiency in the IEs 2 dpb and universally achieved <1% targeted editing. In contrast, the editing efficiencies in regenerated plant tissue were substantially higher, ranging from 1.3-4.7% in one report and 2.4-9.7% in a second report. To investigate this discrepancy between timepoints, we monitored editing efficiency at 14 dpb, 48 dpb, and in the M₀ regenerants in our study. Irrespective of temperature treatment or gRNA sequence, editing frequencies at 48 dpb were considerably higher than at 14 dpb (FIG. 19B). Percentage of tissue edited in the M₀ plant pool was comparable to that at 48 dpb. The observed difference in editing efficiency between earlier timepoints and regenerated M₀ plants was consistent with previous reports.

In mammalian cells, Cas9 was shown to be undetectable 48-72 hours after Cas9-RNP transfection by nucleofection. For this reason, it has been thought that enzymatic degradation of Cas9-RNPs in vivo is rapid and that editing must occur within the first few days of transfection. In the present study, if Cas9-RNPs were fully degraded from the tissue prior to the 14 dpb timepoint, all gene editing would have had to occur before 14 dpb. Consequently, approximately the same number of unique alleles would have been expected to be detected at both 14 dpb and 48 dpb if proliferation of edited and unedited cells occurs at the same rate. On the contrary, consistently higher rates of mutagenesis as well as a greater number of unique alleles at the later timepoints were observed at 48 dpb (FIG. 19B, FIG. 21A, FIG. 22), suggesting that Cas9 may somehow be stabilized for at least 14 days and gradually released within the wheat IEs after biolistic delivery for sustained editing over time. As further evidence in support of this hypothesis, Cas9 protein was detected in 10-embryo tissue samples taken 2, 7, and 14 dpb (FIG. 21B). Taken together, these results indicate that Cas9 is maintained in tissue at least 14 dpb and facilitates sustained and gradual editing of tissue over time when delivered as Cas9-RNP via gold particle bombardment. Further biochemical exploration is necessary to understand the mechanism of this Cas9 stabilization and persistent editing.

Numerous plant protoplast systems have been used for targeted mutagenesis using Cas9-RNPs. Although the method is useful for producing Cas9-RNP edited plants for protoplasts that are amenable to regeneration, most crop plants cannot easily be regenerated in this manner. Though wheat protoplasts are recalcitrant to regeneration through existing methodology, protoplasts in the current study prove to be a beneficial screening system. Cas9-RNP mediated editing rates in protoplasts correlated linearly with editing rates in IEs. Because biolistic Cas9-RNP transfection of IEs requires significant time, energy, resources, and commitment, a means for rational selection of gRNA sequences for optimal editing efficiency is preferred. It is noteworthy that there were major differences in mutation rates for the 5 gRNAs used in this study. Unfortunately, existing predictive software to select gRNAs do not translate upon experimentation. Therefore, when attempting to select the best gRNA to produce the highest rate of stable editing in regenerable IEs, transient protoplast assays can serve as a rapid pipeline to rank gRNAs and forecast editing rates in Cas9-RNP bombarded regenerable tissue.

The calculation of editing efficiency in M₀ regenerants has the potential to be confusing. To be explicit in our analysis, we present editing rates of regenerants in two ways. The percentage of total edited alleles in the M₀ regenerant pool is indicated as “% Tissue edited”, while “% Plants edited” is the percentage of total edited plants among all the M₀ plants (FIG. 19B, FIGS. 23A-23B, FIG. 20). The former is meant to compare overall editing efficiency more fairly across tissue types and timepoints, taking biallelism, homozygosity, and heterozygosity of regenerated plants into consideration. The latter value is more relevant for evaluating the method's ability to produce individual plants with gene edits.

Pi21 was first characterized in rice (Oryza sativa) as a negative regulator of resistance for blast disease. We identified putative orthologs in wheat that consisted of three homoeologous genes. The functionality of wheat Pi21 has not been formally assessed but may potentially play a role in disease susceptibility. Wheat Pi21 was selected as a target to demonstrate the DNA-free Cas9-RNP gene editing method in a gene present in all three diploid subgenomes (AABBDD). Pi21gD was designed to simultaneously target all six alleles. Despite the genetic complexity, we were able to regenerate plants with biallelic or homozygous mutations across all three subgenomes for a full variety of genotypes including two with homozygous triple mutant knockouts within the M₀ generation.

The wheat genes Tsn1 and Snn5 recognize the Parastagonospora nodorum pathogenic effectors SnToxA and SnTox5 respectively. Tsn1 is a gene with resistance gene-like features including protein kinase, nucleotide binding, and leucine-rich repeats, and the ToxA necrotrophic effector is produce by at least three economically important fungal pathogens of wheat. Snn5 belongs to a different class and contains protein kinase and major sperm protein domains, but like Tsn1, it functions as a target for a necrotrophic effector leading to disease susceptibility. Therefore, Tsn1 and Snn5 are practical targets for disruption via DNA-free gene editing. Using DNA-free biolistic delivery of Cas9-RNPs, we successfully generated plants with heterozygous, biallelic, and homozygous mutations within the M₀ generation from a mere ten IEs per treatment. Biallelic and homozygous mutants of Tsn1 and Snn5 were demonstrated to be insensitive to SnToxA and SnTox5 respectively. Due to the high rate of editing, particularly using Snn5 g1 and Snn5 g2 with 30° C. and 37° C. heat treatment, screening of M₀ plants for edits was fully feasible. Contrary to previous reports, a selection scheme can reasonably be foregone with Cas9-RNP mediated editing so long as gRNAs are pre-tested in protoplasts and deemed to be highly effective.

In summary, heat treatment enhancement of Cas9-RNP mediated wheat editing combined with a protoplast-based approach to select optimal gRNAs, and findings that editing is sustained for more than 2 weeks advances this DNA and selection-free gene editing approach in crops. The presented advancement to this technology can be applied to numerous crops that are amenable to particle bombardment and encourages the establishment of tissue culture and regeneration protocols in crop species that are vegetatively propagated.

### Materials and Methods

### Plant Material

The allohexaploid wheat (Triticum aestivum L., 2n=6x=42, AABBDD genomes) cultivar Fielder was used for this study.

### Cas9-gRNA RNP Assembly

Cas9 protein with a C-terminal double nuclear-localization tag (QB3 Macrolab, University of California, Berkeley) and sgRNAs with modifications of 2′-O-Methyl at 3 first and last bases, and 3′ phosphorothioate bonds between first 3 and last 2 bases (Synthego, Menlo Park, CA) were complexed in vitro to form Cas9-gRNA RNPs.

For each protoplast transfection, a 25 μl reaction was assembled. Thoroughly mixed were 10 μg sgRNA, 2.5 μl 10×NEBuffer 3.1 (New England Biolabs, Ipswich, MA), and nuclease-free water. Then, in a drop-wise manner, 10 μg Cas9 was added slowly with constant mixing, followed by 20 min incubation at 37° C.

For each IE biolistic transfection, a 40 ul reaction was assembled. Thoroughly mixed were 6.4 μg sgRNA, 4 μl 10×NEBuffer 3.1, and nuclease-free water. Then, in a drop-wise manner, 12.8 μg Cas9 was added slowly with constant mixing, followed by 20 min incubation at 37° C. The resultant RNP mixtures were stored on ice until transfection.

### Protoplast Isolation and Transfection

Partially etiolated seedlings were used as donor tissue for protoplast isolation. Seeds were surface sterilized in 20% (v/v) bleach and rinsed in sterile water. Seedlings were grown under sterile conditions on wet filter paper in the dark for 12-14 days at 25° C. with exposure to ambient light for 6 hours every 5 days. Wheat protoplasts were isolated from the donor tissue. For each transfection 25 μl of Cas9-gRNA RNP mixture, as defined above, were added to 5×10⁵ protoplasts. PEG-meditated transfection was performed. Protoplasts were harvested 24 hours post-transfection for analysis.

### Gold Particle Preparation for Bombardment

Cas9-RNPs were precipitated onto 0.6 μm gold particles (#1652262, Bio-Rad, Hercules, CA) using the cationic lipid polymer TransIT-2020 (Mirus, Madison, WI). Briefly, for each 30-IE transfection, 40 μl Cas9-RNP mixture, as described above, was mixed gently with 20 μl sterile gold particles (10 μg μl⁻¹ water suspension) and 1 μl TransIT-2020 and incubated on ice for 20 min. The Cas9-RNP coated gold particles were pelleted in a mini microcentrifuge at 2,000 g for 30 s. The supernatant was removed, and the gold particles were resuspended in 20 μl of sterile water by brief sonication. The coated gold particles were immediately applied to 2 macrocarriers (10 μl each) by spotting numerous small drops and allowed to air dry in a laminar flow hood. For a single transfection, each 30-IE set was bombarded twice using the 2 prepared macrocarriers.

### Immature Embryo Bombardment and Regeneration

Plants were grown at 24° C., 16-hour days and 15° C., 8-hour nights under light intensity of 130 μmol m⁻²s⁻¹. Immature seeds containing IEs, sized 1.7-2.2 mm were harvested from wheat spikes 10-13 days after flowering, surface sterilized in 20% (v/v) bleach with one drop of Tween 20 and triple rinsed with sterile water, followed by extraction of the IEs. The IEs were placed on DBC3 media, scutellum side up and incubated overnight at 26° C. prior to biolistic transfection. Four hours prior to bombardment, IEs were placed on 55 mm filter paper in the center of DBC3 osmoticum media containing 0.2 M mannitol and 0.2 M sorbitol. Using two prepared microcarriers holding Cas9-RNP coated gold microparticles, IEs were shot twice using the PDS-100/He gene gun (Bio-Rad, Hercules, CA) with rupture pressure of 1100 psi. The bombarded IEs were transferred from the filter paper directly to the media below and incubated at 26° C., 30° C., or 37° C. for 16 hours. IEs were transferred to standard DBC3 media in dim light (10 mol m⁻² s⁻¹) at 26° C. for 9 weeks with subculturing as needed. Callus tissue originating from each IE was transferred to DBC6 media for regeneration. Resultant plantlets were transferred to rooting media and incubated in high light (90 μmol m⁻² s⁻¹) at 26° C. and grown to 4-6 inches before being transplanted to soil.

### Amplicon Next Generation Sequencing Analysis

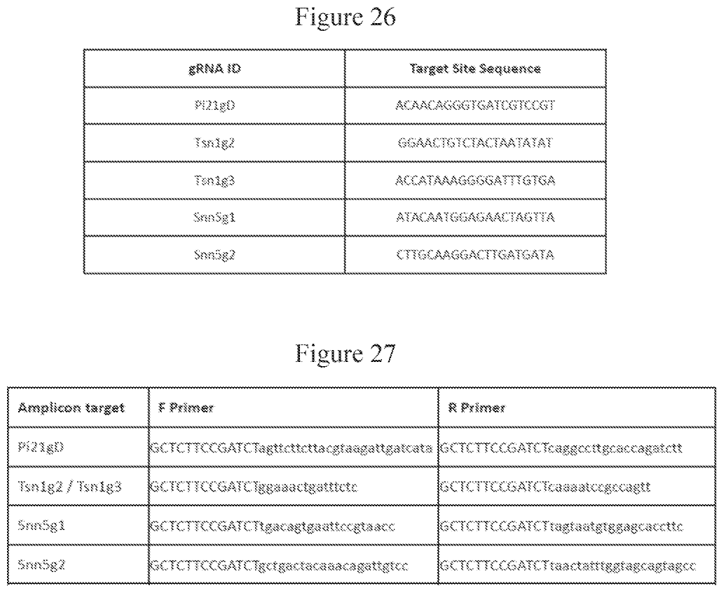

To determine mutation rates by amplicon sequencing, PCR was performed to amplify approximately 225 bp including gRNA target sequences (FIG. 26) using Phusion High Fidelity (New England Biolabs, Ipswich, MA) polymerase. Primers contained a 5′-stub compatible with Illumina NGS library preparation (FIG. 27). PCR products were ligated to Illumina TruSeq adaptors and purified. Libraries were prepared using a NEBNext kit (Illumina, San Diego, CA) according to the manufacturer's guidelines. Samples were deep sequenced on an illumina iSeq at 200 bp paired-end reads to a depth of approximately 10,000 reads per sample Cortado was used to analyze editing outcomes. Briefly, reads were adapter trimmed then merged using overlap to singe reads. These joined reads were then aligned to the target reference sequence. Editing rates are calculated by counting any reads with an insertion or deletion overlapping the cut site or occurring within a 3 bp window on either side of the cut site. SNPs occurring within the window around the cut site are not counted Total edited reads are then divided by the total number of aligned reads to get percent edited.

### Western Blot

Total plant tissue originating from 10 IEs at different timepoints were frozen in LN2, ground to a fine powder by mortar and pestle, and resuspended in 200 μl 2× Laemmli Sample Buffer (Bio-Rad, Hercules, CA) with 2-mercaptoethanol. Samples were boiled for 5 min, and the total soluble protein extracts (25 μl or 40 μl per well) were separated on 4-20% Mini-PROTEAN TGX precast polyacrylamide gels (Bio-Rad, Hercules, CA) and subsequently transferred to a 0.45 μm nitrocellulose membrane (GVS, Sanford, ME). For detection of Cas9 protein, anti-CRISPR/Cas9 C-terminal mouse monoclonal antibody (SAB4200751; Sigma-Aldrich, St. Louis, MO) and ProSignal Dura ECL Reagent (Genesee Scientific, San Diego, CA) were used. PageRuler Plus Prestained Protein Ladder (10-250 kDa, Thermo Fisher, Waltham, MA) was used as a molecular weight marker, and Cas9 protein with a C-terminal double nuclear-localization tag (QB3 Macrolab, University of California, Berkeley) was used as a positive control.

### Production of SnToxA

SnToxA was expressed in the Pichiapastoris yeast strain X33 and cultured in yeast peptone dextrose broth (10 g yeast extract, 20 g peptone, 100 ml 20% dextrose in 900 ml distilled water) for 48 hours at 30° C. Culture filtrate was harvested and filtered through a 0.45 μm HVLP filter membrane (Merk Millipore Ltd., Cork, Ireland) and dialyzed overnight against water using 3.5 kDa molecular weight cut off Snake Skin dialysis tubing (Thermo Scientific, IL, USA). Dialyzed filtrate was loaded onto a HiPrep SpXL 16/10 cation exchange column (GE Healthcare Piscataway, NJ). Unbound protein was washed off the column using a 20 mM sodium acetate (pH 5.0) buffer prior to a gradient elution of SnToxA using a buffer consisting of 300 mM sodium chloride and 20 mM sodium acetate (pH 5.0). Fractions that contained SnToxA were collected and frozen prior to lyophilizing to increase the concentration of SnToxA. Lyophilized samples were dissolved in a buffer consisting of 5 mM MOPS sodium salt (Alfa Aesar, MA, USA) and water, prior to infiltration into the plants.

### Production of SnTox5

P. nodorum strain Sn79+Tox5-3, generated by transforming SnTox5 in to the avirulent P. nodorum strain Sn79-1087, was used to prepare the culture filtrates containing SnTox5. In brief, Sn79+Tox5-3 was grown on V8-potato dextrose agar medium till spores were released from pycnidia. The plates were flooded with 10 ml of sterile distilled water, and 500 μl of spore suspension was used to inoculate 60 ml of liquid Fries medium (5 g ammonium tartrate, 1 g ammonium nitrate, 0.5 g magnesium sulfate, 1.3 g potassium phosphate [dibasic], 3.41 g potassium phosphate [monobasic], 30 g sucrose, 1 g yeast extract in 1000 ml of distilled water). Cultures were grown on an orbital shaker at 100 rpm for a week prior to two weeks of stationary growth under dark conditions at room temperature. Culture filtrates were filtered through a layer of Miracloth (EMD Millipore Corp, MA, USA) and were concentrated 5-fold using Amicon Ultracel—3K centrifugal filters (Merk Millipore Ltd., Cork, Ireland). Culture filtrates were diluted in a 1:1 ratio with sterile water prior to infiltration into the plants.

### Necrotrophic Effector Infiltrations

Infiltrations with SnToxA and SnTox5 containing culture filtrates were conducted. Three infiltrations were performed per plant, and sensitivity was evaluated on a binary scale at 3 days post infiltration.

### Example 7

### RNP-Mediated Gene Editing in Cassava

### Plant Materials and Production of Organized Embryogenic Structures (OES) and Friable Embryogenic Callus (FEC) Tissues

In vitro-cultured cassava (cv. 92/02324) plantlets were maintained in MS2 agar medium for stem elongation and stable growth. Well-developed growing shoots were maintained in growth chambers in Petri dishes or Phytatrays™, three to four shoots per tray, following the conditions described by Taylor et al. (Taylor et al., 2012). OES and FEC used as gene editing target tissues of 92/02324 were generated. Immature leaf lobes were excised from 4- to 6-week-old micropropagated shoot cultures, placed on MS2 50P medium which is Murashige and Skoog basal medium supplemented with 20 g/L sucrose (MS2) plus 50 μM picloram, and incubated in the dark at 28° C. for 4 weeks for induction of OES. OES obtained was excised from the explants using fine forceps and forced through a 1 mm wire mesh. Seven to ten resulting tissue fragments were placed together to form a group. Five to six such groups were cultured in each Petri dish containing Gresshoff and Doy basal medium supplemented with 20 g/L sucrose and 50 μM picloram (GD2 50P) supplemented with 500 μM tyrosine and incubated in the dark for 5 days. After 5 days, tissues were then transferred to fresh medium of the same type and cultured in the dark for a further three to 4 weeks. Small groups of FEC formed after this time were carefully separated from the non-embryogenic tissue using fine forceps and subcultured onto fresh GD2 50P supplemented with tyrosine and mox. After a further 21 days, FEC was maintained on the same media for bombardment with gold particles coated with RNPs.

### Cas9-gRNA RNP Assembly

Cas9 protein with a C-terminal double nuclear-localization tag (QB3 Macrolab, Berkeley) and sgRNAs (IDT, Coralville, IA) were complexed in vitro to form Cas9-gRNA RNPs. For 3 shots for each sample bombardment, a 60 μl reaction was assembled. 9.6 μg sgRNA, 6 μl 10×NEBuffer 3.1, and nuclease free water were thoroughly mixed. Then, in a drop-wise manner, 19.2 μg Cas9 was added slowly with constant mixing, followed by 20 min incubation at 37° C. The resultant RNP mixtures were stored on ice until gold particle prep.

### Gold Particle Preparation for Bombardment

Cas9-RNPs were precipitated onto 0.6 μm gold particles (Bio-Rad, Hercules, CA) using the cationic lipid polymer, TransIT-2020 (Mirus, Madison, WI). Briefly, for 3 shots, 60 l Cas9-RNP mixture, as described above, was mixed gently with 30 μl sterile gold particles (10 μg μl⁻¹ water suspension) and 1.5 μl TransIT-2020 and incubated on ice for 20 min. The Cas9-RNP coated gold particles were pelleted in a mini microcentrifuge at 2,000 g for 30 s. The supernatant was removed, and the gold particles were resuspended in 30 μl of sterile water by brief sonication. The coated gold particles were immediately applied to 3 macrocarriers (10 μl each) by spotting numerous small drops and allowed to air dry in a laminar flow hood. Each sample was bombarded three times using the 3 prepared macrocarriers.

### OES/FEC Bombardment and Regeneration

Four hours prior to bombardment, OES and/or FEC were placed on 55 mm filter paper in the center of MS2 50P/GD2 50P osmoticum media supplemented with 0.2 M mannitol and 0.2 M sorbitol. Using three prepared microcarriers holding Cas9-RNP coated gold microparticles, tissues for each sample were shot three times using the PDS-100/He gene gun (Bio-Rad, Hercules, CA) with rupture pressure of 1100 psi. The bombarded tissues were transferred from the filter paper directly to the media below and incubated at 28° C., 30° C., or 34° C. for 20 hours. Bombarded OES tissues were maintained MS2 50P media for 3 weeks and moved to next step for FEC induction. Bombarded FEC issues were transferred to GD2 50 media in dim light (10 μmol m⁻² s⁻¹) at 28° C. for 8-12 weeks with subculturing as needed. The tissues were transferred to embryo regeneration (Stage 1 medium, MS2 supplemented with 5 μM NAA) for 21 days followed by embryo maturation media (Stage 2, MS2 supplemented with 0.5 μM NAA). Shoots were recovered on MS2 containing 2 μM BAP and maintained on MS2 Agar.

### Amplicon Next Generation Sequencing Analysis

To determine mutation rates for the OES/FEC tissues at 2 weeks after bombardment or the pooled regenerated plantlets 3-4 months after bombardment by amplicon sequencing, PCR was performed with target-specific primers, amplifying approximately 200-300 bp around the cut site using Phusion High Fidelity (New England Biolabs, Ipswich, MA) polymerase. Primers contained a 5′-stub compatible with Illumina NGS library preparation. PCR products were ligated to Illumina TruSeq adaptors and purified. Libraries were prepared using a NEBNext kit (Illumina, San Diego, CA) according to the manufacturer's guidelines. Samples were deep sequenced on an Illumina iSeq at 200-300 bp paired end reads to a depth of approximately 10,000 reads per sample. Cortado was used to analyze editing outcomes. Briefly, reads were adapter trimmed then merged using overlap to single reads. These joined reads were then aligned to the target reference sequence.

### Detection of CRISPR/Cas9 Mutations

Genomic DNA was extracted from leaf tissue of individual plants from the pools (groups) with positive Amplicon sequencing results following a CTAB extraction method. PCR amplifications using sequence specific primers to amplify a fragment within range of the desired mutation were performed in a 25-μl reaction with DreamTaq PCR Master Mix (2×) (Thermo Fisher Scientific, Grand Island, NY). For each PCR reaction, 23 μL were loaded onto a 0.8% agarose gel for electrophoresis. The amplified PCR product was cut, and DNA was extracted from gel samples using the Qiagen QIAquick Gel Extraction Kit (QIAGEN, Chatsworth, CA). The purified DNA samples were used for Sanger sequencing to determine mutation patterns.

### Example 8

### RNP-Mediated Gene Editing in Cacao

### Plant Materials and Production of Donor Tissues for RNP Bombardment

Immature flowers were used to initiate primary somatic embryos of cacao (cvs. INIAPG-038, Matina 1-6, and CCN-51). Cotyledon tissues were generated from in vitro-cultured cotyledon primary somatic embryos and used for RNP bombardment.

### Cas9-gRNA RNP Assembly

Cas9 protein with a C-terminal double nuclear-localization tag (QB3 Macrolab, Berkeley) and sgRNAs (IDT, Coralville, IA) were complexed in vitro to form Cas9-gRNA RNPs. For 3 shots for each sample bombardment, a 60 μl reaction was assembled. 9.6 μg sgRNA, 6 μl 10×NEBuffer 3.1, and nuclease free water were thoroughly mixed. Then, in a drop-wise manner, 19.2 μg Cas9 was added slowly with constant mixing, followed by 20 min incubation at 37° C. The resultant RNP mixtures were stored on ice until gold particle prep.

Gold particle Preparation for Bombardment Cas9-RNPs were precipitated onto 0.6 μm gold particles (Bio-Rad, Hercules, CA) using the cationic lipid polymer, TransIT-2020 (Mirus, Madison, WI). Briefly, for 3 shots, 60 μl Cas9-RNP mixture, as described above, was mixed gently with 30 μl sterile gold particles (10 μg μl⁻¹ water suspension) and 1.5 μl TransIT-2020 and incubated on ice for 20 min. The Cas9-RNP coated gold particles were pelleted in a mini microcentrifuge at 2,000 g for 30 s. The supernatant was removed, and the gold particles were resuspended in 30 μl of sterile water by brief sonication. The coated gold particles were immediately applied to 3 macrocarriers (10 μl each) by spotting numerous small drops and allowed to air dry in a laminar flow hood. For a single transfection, each 30-IE set was bombarded twice using the 3 prepared macrocarriers.

OES/FEC Bombardment and Regeneration Four hours prior to bombardment, cotyledon tissues derived from primary somatic embryos were cut into 4-10 mm in size and placed on 55 mm filter paper in the center of secondary callus growth (SCG) osmoticum media supplemented with 0.2 M mannitol and 0.2 M sorbitol. Using three prepared microcarriers holding Cas9-RNP coated gold microparticles, tissues for each sample were shot three times using the PDS-100/He gene gun (Bio-Rad, Hercules, CA) with rupture pressure of 1100 psi. The bombarded tissues were incubated at 28° C., 30° C., or 34° C. for 20 hours, transferred to SCG media and incubated in the dark at 28° C. for 8-12 weeks with subculturing as needed. The tissues were then transferred to ED4 (embryo development medium containing 4% sucrose) for 2-4 weeks then subsequently on ED3 (embryo development medium containing 3% sucrose). When germinating embryos were approximately 15-20 mm in length, the conversion process began by placing them onto ED6 medium (embryo development containing 6% sucrose); they were exposed to low levels of light-emitting diode (LED) light, 10-30 μmol m⁻² s⁻¹. These embryos were incubated at 28° C. and the Petri dishes were then wrapped with 3M Micropore tape (St. Paul, MN) to allow for gas exchange. After 2-3 weeks on ED6 the embryos were transferred to embryo development-light (EDL) media and then maintained on EDL with transfers every two weeks. As the cultures developed, they were slowly exposed to higher LED light intensities, 30-60 μmol m⁻² s⁻¹. When the plantlets were 2.5 cm tall, they were transferred to a Phytatray™ II (Sigma-Aldrich, St. Louis, MO) containing 100 mL of rooting medium and sealed with 3M Micropore tape.

### Amplicon Next Generation Sequencing Analysis

To determine mutation rates for the cotyledon tissues at 2 weeks after bombardment or pooled callus tissues/somatic embryos 2-3 months after bombardment by amplicon sequencing, PCR was performed with target-specific primers, amplifying approximately 200-300 bp around the cut site using Phusion® High Fidelity (New England Biolabs, Ipswich, MA) polymerase. Primers contained a 5′-stub compatible with Illumina NGS library preparation. PCR products were ligated to Illumina TruSeq adaptors and purified. Libraries were prepared using a NEBNext kit (Illumina, San Diego, CA) according to the manufacturer's guidelines. Samples were deep sequenced on an Illumina iSeq at 200-300 bp paired end reads to a depth of approximately 10,000 reads per sample. Cortado was used to analyze editing outcomes. Briefly, reads were adapter trimmed then merged using overlap to single reads. These joined reads were then aligned to the target reference sequence.

### Detection of CRISPR/Cas9 Mutations

Genomic DNA was extracted from somatic embryo or leaf tissue of individual plantlets from the pools with positive Amplicon sequencing results following a CTAB extraction method. PCR amplifications using sequence specific primers to amplify a fragment within range of the desired mutation were performed in a 25-μl reaction with DreamTaq PCR Master Mix (2×) (Thermo Fisher Scientific, Grand Island, NY). For each PCR reaction, 23 μL were loaded onto a 0.8% agarose gel for electrophoresis. The amplified PCR product was cut, and DNA was extracted from gel samples using the Qiagen QIAquick Gel Extraction Kit (QIAGEN, Chatsworth, CA). The purified DNA samples were used for Sanger sequencing to determine mutation patterns.

### Example 9

### Optimal Dispersal of Protein-, RNP- or RNP/DNA-Coated Gold Microparticle Suspension Per Volume to Improve Drying Time and Microparticle-Covering Area

### Plant Materials and Production of Embryogenic Callus Tissues

Seeds of Triticum aestivum L. cv. Fielder were sown weekly and grown in growth chambers under 16-hr days at 24° C., and 8-hr nights at 15° C. Light levels were set to approximately 130 μmol m⁻² s⁻¹ at head height. Immature spikes were harvested 10-14 days post flowering with an immature embryo (IE) sized 1.7-2.2 mm. Immature spikes were collected up to 3 days pre bombardment and stored at 4-6° C. One day prior to bombardment, immature seeds were harvested from immature spikes and surface-sterilized using 20% (v/v) bleach (8.25% sodium hypochlorite) plus one drop of Tween 20 for 15 minutes before triple rinsing with sterile water. IEs were then isolated and placed scutellum side up on DBC3 medium (Cho et al. 1998) and incubated at 28° C. overnight.

### Gold Particle Preparation for Bombardment

Purified DsRED protein (Takara Bio, San Jose, CA) was precipitated onto 0.6 μm gold particles (Bio-Rad, Hercules, CA) with the cationic lipid polymer, TransIT-2020 (Mirus, Madison, WI). Briefly, for 4 shots, 25.6 μl of purified DsRED protein at 1 μg/μl and 54.4 μl sterile water was mixed gently with 40 μl sterile gold microparticles (20 μg μl⁻¹ water suspension)+/−2 μl TransIT-2020. The DsRED gold microparticles were pelleted in a mini microcentrifuge at 2,000 g for 30 sec. The supernatant was removed, and the gold particles were resuspended in 40 μl of sterile water by brief sonication. The coated gold particles were immediately applied to 4 macrocarriers (10 μl each) with 4 different treatments described in the legend for Table 4 and allowed to air dry in a laminar flow hood.

Cas9 RNPs were precipitated onto 0.6 μm gold particles with TransIT-2020 as previously described with modifications. Briefly, for 6 shots, 120 μl of Cas9-RNP mixture was mixed gently with 60 μl sterile gold particles (20 μg μl⁻¹ water suspension) and 3.0 μl TransIT-2020. The Cas9 RNPs gold particles were pelleted in a mini microcentrifuge at 2,000 g for 30 sec. The supernatant was removed, and the gold particles were resuspended in 60 μl of sterile water by brief sonication. The coated gold particles were immediately applied to 6 macrocarriers (10 μl each) 6 different treatments described in the legend for Table 4 and allowed to air dry in a laminar flow hood.

Cas9 RNPs+DsRED DNA were precipitated onto 0.6 μm gold particles with TransIT-2020 as previously described with modifications. Briefly, for 4 shots, 80 μl of Cas9-RNP mixture containing 0.67 ug of the pAct1IDsRED plasmid (please see Example #1) was mixed gently with 40 μl sterile gold particles (20 μg μl⁻¹ water suspension) and 2.0 μl TransIT-2020. The Cas9 RNPs+DsRED DNA gold particles were pelleted in a mini microcentrifuge at 2,000 g for 30 sec. The supernatant was removed, and the gold particles were resuspended in 40 μl of sterile water by brief sonication. The coated gold particles were immediately applied to 4 macrocarriers (10 μl each) 4 different treatments described in the legend of Table 4 and allowed to air dry in a laminar flow hood.

After loading 10 μl of gold particle suspension to each microcarrier, time needed to dry particle suspension was measured.

### DsRED Protein, RNP or RNP+DNA Bombardment

Four hours prior to bombardment, IEs were placed in the center of a Petri dish containing DBC3 osmoticum media supplemented with 0.2 M mannitol and 0.2 M sorbitol. Using prepared microcarriers holding RNP/DsRED DNA plasmid-coated gold microparticles, IEs for each sample were shot once using the PDS-100/He gene gun (Bio-Rad, Hercules, CA) with rupture pressure of 1100 psi (Table 4), incubated at 28° C. for 20 hours. Bombarded IEs were then transferred to DBC3 media without osmoticum and incubated in the dark 28° C.

In order to test the gold microparticle distribution patterns and DsRED protein- or RNP-coated gold microparticles were bombarded directly onto DBC3 medium solidified with Phytagel without plant tissues.

### Fluorescent Visualization

Fluorescent images of IEs were visualized to evaluate # of DsRED foci and distribution/covering area of DsRED foci on individual IEs using a fluorescent Leica M165 FC stereomicroscope, equipped with Leica DFC7000 T (JH Technologies, Fremont, CA), using two microscopic filters, brightfield and ET DSR with 545 nm excitation and 620 nm emission.

The distribution of gold microparticles by each treatment was also visualized by naked eyes and Leica M165 FC stereomicroscope with brightfield lights to evaluate the distribution/covering area of gold microparticles directly bombarded onto solid DBC3 media.

### Results

It took only 20-25 min to air dry 10 μl of DsRED protein- or RNP-coated gold particle suspension completely when 10 μl of particle suspension was spotted with multiple drops (15 or more drops) onto a microcarrier while it took 66 min with 1 drop spotting (Table 4). Others reported a similar result where they allowed to air-dry at 22° C. for 16 hours when 12 μl of DsRed protein solution was spread directly onto the macrocarrier set. In the present study, spreading multiple drops of particle suspension with a pipet tip at the time of initial drying of part of drops significantly reduced drying time to 15 min and evenly covered all circled target areas of the macrocarrier disk with gold microparcticles (Table 4). Spreading 1 drop of particle suspension also reduced drying time of RNP-coated gold particles from 66 min to 57 min and it covered macrocarrier-covering area almost twice compared to 1 drop spotting without spreading.

Multiple drop spotting distributed particles more evenly in the solid DBC3 media after bombardment while single drop spotting concentrated gold particles in the center of the particle covering area (Table 4). Spreading treatment of multiple drops on the microcarrier disk produced even more unform distribution of gold particles regardless of # of drops.

TABLE 4
Optimal Dispersal of Protein- or RNP-coated Gold Microparticle Suspension
per Volume to Improve Drying Time and Microparticle-covering Area

| | # of protein or RNP drops | | | |
|---|---|---|---|---|
| | 1 drop + | 2 | multi | multi drops + |
| DsRED | spread^{c} | drops^{d} | drops^{f} | spread^{g} |
| Time needed to dry particle | 55 | 45 | 25 | 15 |
| suspension (min) | | | | |
| Particle covering area after | +(+) | +(+) | ++ | ++(+) |
| bombardment^{a} | | | | |

| | 1 | 1 drop + | 2 | 3 | multi | multi drops + |
|---|---|---|---|---|---|---|
| RNP | drop^{b} | spread | drops | drops^{e} | drops | spread |
| Time needed to dry particle | 66 | 57 | 59 | 39 | 20 | 15 |
| suspension (min) | | | | | | |
| Particle covering area after | + | +(+) | +(+) | ++ | ++ | ++(+) |
| bombardment | | | | | | |

^{a}Distribution/covering area of gold microparticles directly bombarded onto solid DBC3 media
^{b}One drop of 10 μl microparticle suspension was spotted on a macrocarrier disk
^{c}One drop of 10 μl microparticle suspension was spotted on a macrocarrier disk and then spread out with a pipet tip at the time of initial drying of the drop
^{d}Two drops of 5 μl microparticle suspension were spotted on a macrocarrier disk
^{e}Three drops of microparticle suspension (10 μl in total) were spotted on a microcarrier disk
^{f}>15 drops of microparticle suspension (10 μl in total) were spotted on a microcarrier disk
^{g}>15 drops of microparticle suspension (10 μl in total) were spotted on a microcarrier disk and then spread out with a pipet tip at the time of initial drying of part of drops

In order to visualize the delivery of RNP by particle bombardment, DsRED plasmid was added to the gold particle suspension. Time needed to dry microparticle suspension for multiple drops was 20 min while 1 drop needed 60 min (Table 5). Two drops and multi drop spotting showed increased # of DsRED-expressing foci per IE compared to 1 drop. Spreading of multiple drops showed the best results with more uniform distribution of particle delivery in terms of # of DsRED expressing foci.

TABLE 5
Optimal Dispersal of RNP/DsRED DNA plasmid-coated Gold Microparticle Suspension
per Volume to Improve Drying Time and Macroparticle-covering Area/Pattern

| | # RNP + DsRED DNA drops | | | | | | | | | | | | | | | |
|---|---|---|---|---|---|---|---|---|---|---|---|---|---|---|---|---|
| | 1 drop + spread^{c} | | | | 2 drops^{d} | | | | Multi drops^{e} | | | | Multi drops + spread^{f} | | | |
| Time needed to dry | 60 | | | | 40 | | | | 20 | | | | 15 | | | |
| microparticle | | | | | | | | | | | | | | | | |
| suspension (min) | | | | | | | | | | | | | | | | |
| # of DsRED-expressing | >200 | 50-200 | 20-50 | <20 | >200 | 50-200 | 20-50 | <20 | >200 | 50-200 | 20-50 | <20 | >200 | 50-200 | 20-50 | <20 |
| foci per IE^{a} | | | | | | | | | | | | | | | | |
| #IEs^{b} | 11 | 10 | 5 | 14 | 22 | 6 | 13 | 9 | 19 | 9 | 14 | 8 | 23 | 12 | 10 | 5 |

^{a}JE: immature embryo
^{b}Fifty IEs were bombarded per microparticle spotting treatment
^{c}One drop of 10 μl microparticle suspension was spotted on a macrocarrier disk and then spread out with a pipet tip at the time of initial drying of the drop
^{d}Two drops of 5 μl microparticle suspension were spotted on a macrocarrier disk
^{e}>15 drops of microparticle suspension (10 μl in total) were spotted on a microcarrier disk
^{f}>15 drops of microparticle suspension (10 μl in total) were spotted on a microcarrier disk and then spread out with a pipet tip at the time of initial drying of part of drops

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of biolistically delivering a substance to plant tissue comprising
(a) applying a rupture or bombardment pressure of about 100 psi to about 3000 psi to a macrocarrier disk carrying at least one drop of coated microparticles in order to bombard the coated microparticles into the plant tissue to form bombarded plant tissue, wherein the microparticles are about 0.1 μm to about 2.0 μm in size and wherein the microparticles are in a volume of microparticle suspension of about 0.1 μl to about 50 μl; and
(b) culturing the bombarded plant tissue on growth media in optimal incubation conditions for each substance.

2. The method of claim 1, wherein the about 0.1 μl to about 50 μl volume of microparticle suspension is present as one to 100 spots on the macrocarrier.

3. The method of claim 1, wherein the about 0.1 μl to about 50 μl volume of microparticle suspension is present as about 15-50 spots.

4. The method of claim 2 or 3, wherein the spots are dispersed over the macrocarrier disk surface.

5. The method of any one of claims 2-4, wherein the spots are spread out over the macrocarrier disk.

6. The method of claim 5, wherein the spreading is performed using a spatula, pipette tip, or other mechanical device.

7. The method of 5 or 6, wherein the macrocarrier disk is dried for about 2 minutes before the spots are spread out.

8. The method of any one of claims 1-7, wherein the macrocarrier disk is dried or lyophilized prior to the application of the rupture or bombardment pressure.

9. The method of any one of claims 1-8, wherein the plant tissue is an immature embryo (IE), callus tissue, somatic embryo, zygotic embryo, meristem tissue, pollen, cotyledon, leaf, stem, or root tissue.

10. The method of any one of claims 1-8, wherein the plant is a crop plant.

11. The method of claim 10, wherein the crop plant is wheat, rice, maize, soybean, pepper, alfalfa, sunflower, cassava, cacao, banana, potato, sweet potato, strawberry, grape, poplar, coffee, walnut, almond, peach, nectarine, plum, apricot, apple, pear, persimmon, blueberry, blackberry, raspberry, or a fruit tree.

12. The method of any one of claims 1-11, wherein the microparticles are about 0.1 μm to about 2.0 μm in size.

13. The method of any one of claims 1-11, wherein the microparticles are about 0.4 to about 1.6 μm in size.

14. The method of any one of claims 1-11, wherein the microparticles are about 0.5 μm to about 0.7 μm in size.

15. The method of any one of claims 1-11, wherein the microparticle is about 0.6 μm in size.

16. The method of any one of claims 1-15, wherein the rupture pressure is about 100 psi to about 3000 psi.

17. The method of any one of claims 1-15, wherein the rupture pressure is about 500 psi to about 2300 psi.

18. The method of any one of claims 1-15, wherein the rupture pressure is about 600 psi to about 2200 psi.

19. The method of any one of claims 1-15, wherein the rupture pressure is about 650 psi to about 1350 psi.

20. The method of any one of claims 1-15, wherein the rupture pressure is about 1100 psi.

21. The method of any one of claims 1-20, wherein the microparticle suspension is in a volume of about 1 μl to about 50 μl per bombardment.

22. The method of any one of claims 1-20, wherein the microparticle suspension is in a volume of about 10 μl to about 30 μl per bombardment.

23. The method of any one of claims 1-20, wherein the number of bombardments is about 1 and 20 the per plant tissue sample.

24. The method of any one of claims 1-20, wherein the number of bombardments is about 2 and 5 the per plant tissue sample.

25. The method of any one of claims 1-20, wherein the number of bombardments is about 2 the per plant tissue sample.

26. The method of any one of claims 1-25, wherein the substance on the microparticle comprises nucleic acids.

27. The method of any one of claims 1-25, wherein the substance on the microparticle comprises a protein of interest.

28. The method of any one of claims 1-25, wherein the substance on the microparticle comprises Cas9 protein.

29. The method of any one of claims 1-25, wherein the substance on the microparticle comprises sgRNA-Cas9 ribonucleoproteins (RNPs).

30. The method of any one of claims 1-29, wherein the incubating of the bombarded plant tissue is performed at about 26° C. to about 40° C.

31. The method of any one of claims 1-29, wherein the incubating of the bombarded plant tissue is performed at about 30° C. to about 37° C.

32. The method of any one of claims 1-29, wherein the incubating of the bombarded plant tissue is performed at about 34° C.

33. The method of any one of claims 1-29, wherein the incubating of the bombarded plant tissue is performed for about 1 hour to about 180 days post-bombardment.

34. The method of any one of claims 1-33, wherein the incubating of the bombarded plant tissue is performed for about 24 hours to about 30 days post-bombardment.

35. The method of any one of claims 1-33, wherein the incubating of the bombarded plant tissue is performed for about 2 days to about 14 days post-bombardment.

36. The method of any one of claims 1-33, wherein the incubating of the bombarded plant tissue is performed for about 7 days post-bombardment.

37. The method of any one of claims 1-33, wherein the incubating of the bombarded plant tissue is performed for about 3 days post-bombardment.

38. A method of delivering a substance to plant tissue comprising
(a) biolistically delivering microparticles coated with the substance to the plant tissue to generate bombarded plant tissue,
wherein the microparticles are biolistically delivered with a gene gun,
wherein two or more drops of microparticle suspension spread over the inner circle of the macrocarrier disk before drying or lyophilization,
wherein a drop of microparticle suspension is in a volume of about 0.1 μl to about 50 μl,
wherein the microparticles are about 0.1 μm to about 2.0 μm in size, and
wherein the biolistical delivering is performed at a rupture or bombardment pressure of about 100 psi to about 3000 psi, and
(b) culturing the bombarded plant tissue on growth media in optimal incubation conditions for each substance.

39. The method of claim 38, wherein the plant tissue is an immature embryo (IE), callus tissue, somatic embryo, zygotic embryo, meristem tissue, pollen, cotyledon, leaf, stem, or root tissue.

40. The method of claim 38, wherein the plant is a crop plant.

41. The method of claim 40, wherein the crop plant is wheat, rice, maize, soybean, pepper, alfalfa, sunflower, cassava, cacao, banana, potato, sweet potato, strawberry, grape, poplar, coffee, walnut, almond, peach, nectarine, plum, apricot, apple, pear, persimmon, blueberry, blackberry, raspberry, or a fruit tree.

42. The method of any one of claims 38-41, wherein the microparticles are about 0.1 μm to about 2.0 μm in size.

43. The method of any one of claims 38-41, wherein the microparticles are about 0.4 to about 1.6 μm in size.

44. The method of any one of claims 38-41, wherein the microparticles are about 0.5 μm to about 0.7 μm in size.

45. The method of any one of claims 38-41, wherein the microparticle is about 0.6 μm in size.

46. The method of any one of claims 38-45, wherein the rupture pressure is about 100 psi to about 3000 psi.

47. The method of any one of claims 38-45, wherein the rupture pressure is about 500 psi to about 2300 psi.

48. The method of any one of claims 38-45, wherein the rupture pressure is about 600 psi to about 2200 psi.

49. The method of any one of claims 38-45, wherein the rupture pressure is about 650 psi to about 1350 psi.

50. The method of any one of claims 38-45, wherein the rupture pressure is about 1100 psi.

51. The method of any one of claims 38-50, wherein the microparticle suspension is in a volume of about 1 μl to about 50 μl per bombardment.

52. The method of any one of claims 38-50, wherein the microparticle suspension is in a volume of about 10 μl to about 30 μl per bombardment.

53. The method of any one of claims 38-50, wherein the number of bombardments is about 1 and 20 the per plant tissue sample.

54. The method of any one of claims 38-50, wherein the number of bombardments is about 2 and 5 the per plant tissue sample.

55. The method of any one of claims 38-50, wherein the number of bombardments is about 2 the per plant tissue sample.

56. The method of any one of claims 38-55, wherein the substance on the microparticle comprises nucleic acids.

57. The method of any one of claims 38-55, wherein the substance on the microparticle comprises a protein of interest.

58. The method of any one of claims 38-55, wherein the substance on the microparticle comprises Cas9 protein.

59. The method of any one of claims 38-55, wherein the substance on the microparticle comprises sgRNA-Cas9 ribonucleoproteins (RNPs).

60. The method of any one of claims 38-59, wherein the incubating of the bombarded plant tissue is performed at about 26° C. to about 40° C.

61. The method of any one of claims 38-59, wherein the incubating of the bombarded plant tissue is performed at about 30° C. to about 37° C.

62. The method of any one of claims 38-59, wherein the incubating of the bombarded plant tissue is performed at about 34° C.

63. The method of any one of claims 38-59, wherein the incubating of the bombarded plant tissue is performed for about 1 hour to about 180 days post-bombardment.

64. The method of any one of claims 38-63, wherein the incubating of the bombarded plant tissue is performed for about 24 hours to about 30 days post-bombardment.

65. The method of any one of claims 38-63, wherein the incubating of the bombarded plant tissue is performed for about 2 days to about 14 days post-bombardment.

66. The method of any one of claims 38-63, wherein the incubating of the bombarded plant tissue is performed for about 7 days post-bombardment.

67. The method of any one of claims 38-63, wherein the incubating of the bombarded plant tissue is performed for about 3 days post-bombardment.

68. A method of delivering RNPs to plant tissue comprising
(a) biolistically delivering microparticles coated with the substance to the plant tissue to generate bombarded plant tissue, wherein the microparticles are biolistically delivered with a gene gun,
(b) the incubating of the bombarded plant tissue is performed at about 26° C. to about 40° C., and
(c) culturing the bombarded plant tissue on growth media.
